(19) United States
(12) Patent Application Publication
(10) Pub. No.: US 2018/0207080 A1
(43) Pub. Date: Jul. 26, 2018

(54) COMPOSITIONS OF FLUORINATED SURFACTANTS AND ANTIOXIDANTS

(71) Applicant: MERCK PATENT GMBH, DARMSTADT (DE)

(72) Inventors: Manuela LOCH, Darmstadt (DE); Reiner FRIEDRICH, Seeheim-Jugenheim (DE)

(73) Assignee: MERCK PATENT GMBH, DARMSTADT (DE)

(21) Appl. No.: 15/744,595

(22) PCT Filed: Jun. 16, 2016
(86) PCT No.: PCT/EP2016/001009

§ 371 Date: Jan. 12, 2018

(30) Foreign Application Priority Data
Jul. 14, 2015 (EP) 15176709.2

(51) Int. Cl. A61K 8/69; A61Q 19/04; A61K 8/46; A61K 8/37; A61Q 5/02; A61Q 5/12

(52) U.S. Cl. CPC A61K 8/69; A61Q 19/04; A61K 8/46; A61K 2800/522; A61Q 5/02; A61Q 5/12; A61K 8/37

(57) ABSTRACT

The invention relates to the use of specific fluorosurfactants for increasing the efficacy of antioxidants, in particular in cosmetic, dermatological preparations and medicinal products, and to specific mixtures and preparations

The invention relates to the use of specific fluorosurfactants for increasing the efficacy of antioxidants, in particular in cosmetic, dermatological preparations and medicinal products, and to specific mixtures and preparations.

A multiplicity of surfactants is known for use in cosmetic preparations.

EP 2349989 describes, for example, alkylsulfosuccinate monoesters and mixtures of alkylsulfosuccinate monoesters as emulsifiers for cosmetic preparations, where the alkyl groups are linear or branched, saturated or unsaturated and have 6 to 22 C atoms. The compounds are furthermore described as advantageous for the stabilisation or dispersion of solid ingredients or active compounds, for example of pigments or UV filters, in cosmetic preparations.

A further class of surfactants comprises fluorosurfactants. Fluorosurfactants have an outstanding ability to reduce the surface tension, which is utilised, for example, in the hydrophobicisation of surfaces, for example of textiles, paper, glass, building materials or adsorbents. Their use as interface promoter or emulsifier or viscosity reducer in cosmetic preparations is likewise known.

Specific applications of sulfosuccinates and/or sulfotricarballylates having various fluorinated side chains are described in U.S. Pat. No. 4,968,599 and U.S. Pat. No. 4,988,610 and U.S. Pat. No. 6,890,608 and in A. R. Pitt et al., Colloids and Surfaces A: Physicochemical and Engineering Aspects, 1996, 114, 321-335; A. R. Pitt, Progr. Colloid Polym. Sci, 1997, 103, 307-317 and Z.-T. Liu et al., Ind. Eng. Chem. Res. 2007, 46, 22-28. Further fluorosurfactants, in particular succinates and tricarballylates containing fluorinated alkyl groups, are described in WO 2009/149807, WO 2010/003567, WO 2010/149262, WO 2011/082770 and WO 2012/084118.

In WO 2010/149262, the fluorosurfactants described therein are described, inter alia, as UV stabilisers for polymeric materials (plastics) or for filling compositions.

The object of the present invention consisted in the provision of further areas of application of known fluorosurfactants which bring about improved properties of the preparation, in particular in cosmetic or dermatological preparations or medicinal products.

Surprisingly, it has now been found that specific tricarballylates containing fluorinated alkyl groups in combination with antioxidants increase the efficacy of the latter. The increase in the efficacy can also be described as a boost effect.

The present invention therefore relates firstly to the use of compounds of the formula (I)

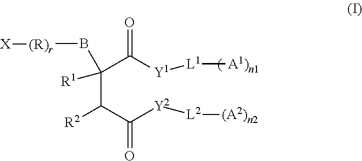

where

X is a hydrophilic group,

R is linear or branched alkylene having 1 to 12 C atoms, where one or more non-adjacent C atoms may be replaced by O, S and/or N,

r is 0 or 1,

B is a single bond, —O—, —NH—, —NR′—, —CH₂—, —C(O)—O—, —S—, —CH₂—O—, —O—C(O)—, —O—C(O)—O—, —N—C(O)—, —C(O)—N—, —O—C(O)—N—, —N—C(O)—N—, —Si(R′)₂—, —Si(R′)₂—O—, —O—SO₂— or —SO₂—O—,

R′ is a linear or branched alkyl group having 1 to 12 C atoms,

R¹ and R², independently of one another, are hydrogen or —CH₂—COY³-L³-(A³)ₙ₃,

Y¹, Y² and Y³, independently of one another, are O, S or N,

L¹, L² and L³, independently of one another, are linear or branched alkylene having 1 to 12 C atoms, where one or more non-adjacent C atoms may be replaced by O, S and/or N,

A¹, A² and A³, independently of one another, are hydrogen or a group of the structure —Zⁱ(CR³R⁴)ₘᵢRfⁱ, where i denotes 1, 2 or 3,

Zⁱ is O, S or N and is bonded to a C atom of Lⁱ or is a single bond,

R³ and R⁴, independently of one another, are hydrogen or a straight-chain or branched alkyl group having 1 to 12 C atoms,

Rfⁱ is a fluorine-containing radical,

n1, n2 and n3, independently of one another, are 1, 2 or 3,

m1, m2 and m3, independently of one another, are 0, 1, 2, 3, 4 or 5, and the compounds of the formula (I) contain at least one Rfⁱ group, for increasing the efficacy of antioxidants.

The abbreviation Lⁱ here stands in place of the abbreviations L¹, L² and/or L³.

If the term “where one or more non-adjacent C atoms may be replaced by O, S and/or N” is used, this means that one or more non-adjacent C atoms may be replaced by O, S, NH and/or NR′, where R′ has a meaning given above or given as preferred.

Straight-chain or branched alkyl groups having 1 to 4, 1 to 6, 1 to 8 or 1 to 12 C atoms conform to the formula CₚH₂ₚ₊₁ where p=1, 2, 3 or 4, or 1, 2, 3, 4, 5 or 6, or 1, 2, 3, 4, 5, 6, 7 or 8, or 1, 2, 3, 4, 5, 6, 7, 8, 9, 10, 11 or 12, for example methyl, ethyl, i-propyl, propyl, butyl, i-butyl or tert-butyl, furthermore also pentyl, 1-, 2- or 3-methylbutyl, 1,1-, 1,2- or 2,2-dimethylpropyl, 1-ethylpropyl or hexyl, heptyl, octyl, furthermore also nonyl, decyl, undecyl or dodecyl.

If an alkyl group is not designated in greater detail, it is a straight-chain alkyl group. In perfluoroalkyl groups, in each case all H atoms of an alkyl group have been replaced by F atoms.

Linear or branched alkylene having 1 to 12 C atoms conforms to the formula CₚH₂ₚ and is derived from the straight-chain or branched alkyl groups described above.

The efficacy of the antioxidants is improved, in particular, in cosmetic or dermatological preparations or in medicinal products.

The efficacy of the antioxidants is particularly preferably improved in cosmetic preparations.

The effect of the increase in the efficacy of antioxidants, for example evident from the prevention of the decomposition of beta-carotene after irradiation with, for example, sun-like light, is surprisingly observed for various types of antioxidant. The fluorosurfactants themselves do not exhibit any efficacy here, as explained in the example part.

The fluorosurfactants of the formula (I) to be used in accordance with the invention are preferably built up from a plurality of short-chain perfluoroalkyl groups with anionic, cationic, nonionic and amphoteric groups.

In particular, branched fluorosurfactants having short-chain perfluoro-alkyl chains can lead to an improved ecotoxicological profile, since such compounds are non-toxic and do not exhibit bioaccumulation or inhalation toxicity. Their use in the cosmetics field is particularly preferred owing to this property.

Preferred compounds of the formula (I) are those which contain two or three Rfⁱ groups. However, compounds containing at least four Rfⁱ groups are also possible, preferably containing four, six or nine Rfⁱ groups.

The fluorinated groups Rfⁱ used are preferably branched or unbranched, fluorine-containing alkyl radicals, in particular perfluorinated alkyl radicals. Particular preference is given to fluorine-containing alkyl radicals having 1 to 10, preferably 1 to 6, in particular 1 to 4, C atoms. Especial preference is given to the use of perfluorinated Rfⁱ groups having 1 to 6, in particular 1 to 4, C atoms. Rf¹, Rf² and Rf³ preferably have the same meaning.

R¹ and R² are preferably not simultaneously —CH₂—COY³-L³-(A³)ₙ₃.

Preferred compounds of the formula (I) are in addition those in which Y¹, Y² and Y³ preferably denote O or N, in particular O. Y¹ and Y² or Y¹, Y² and Y³ preferably have the same meaning.

The groups Rfⁱ are bonded to a group L¹, L² or L³ via a —Zⁱ(CR³R⁴)ₘᵢ group. Zⁱ here preferably stands for O or N, in particular for O. Preference is given to compounds in which all Zⁱ are identical.

Preferred compounds of the formula (I) are those in which n1, n2 and n3 are preferably, independently of one another, 1 or 2.

Particularly preferred compounds of the formula (I) are those in which n1, n2 and n3 are preferably, independently of one another, 1.

Preferred compounds of the formula (I) are also those in which m1, m2 and m3 are preferably, independently of one another, 0, 1, 2, 3 or 4, in particular 0 or 1.

Preferred compounds of the formula (I) are also those in which R³ and R⁴ are, independently of one another, hydrogen or a linear or branched alkyl group having 1 to 6 C atoms, in particular 1-4 C atoms. R³ and R⁴ preferably stand, independently of one another, for hydrogen or an unbranched alkyl group having 1 to 3 C atoms. Preference is given to compounds in which R³ or R⁴ is equal to hydrogen. Preference is given to compounds in which all Zⁱ, R³, R⁴ and mi in each case have the same meaning.

L¹, L² and L³ can preferably, independently of one another, be linear or branched alkylene having 1 to 10 C atoms. In particular, L¹, L² and L³ are, independently of one another, linear or branched alkylene having 2 to 8 C atoms. L¹, L² and L³ are, independently of one another, particularly preferably linear or branched alkylene having 2 to 6 C atoms. One or more non-adjacent C atoms of the groups L¹, L² and L³ may preferably be replaced by O or N, preferably by O. In a preferred embodiment variant of the compounds of the formula (I), L¹ and L² are identical. If L³ is also present, L¹ and L² or L¹ and L³ or L² and L³ may preferably be identical. In a particularly preferred embodiment of the compounds of the formula (I), all groups L¹, L² and L³ are identical.

Particular preference is given to compounds of the formula (I) in which at least one group Lⁱ stands for —(CR⁵R⁶)_{ci}(CR⁷R⁸)_{c′i}—, where the indices ci and c′i stand, independently of one another, for 0 to 10, R⁵ to R⁸ stand, independently of one another, for hydrogen or a branched or unbranched alkyl group having 1 to 6 C atoms, with the condition that ci and c′i do not simultaneously stand for 0. The sum ci and c′i is preferably 1, 2, 3, 4, 5, 6, 7, 8, 9 or 10. ci and c′i are particularly preferably 1.

Especial preference is given to compounds of the formula (I) in which, in at least one group Lⁱ, the group R⁵ denotes an alkyl group having 1 to 6 C atoms, in particular denotes an alkyl group having 1 or 2 C atoms, and the groups R⁶ and R⁷ and R⁸ stand for hydrogen.

Preference is furthermore also given to compounds of the formula (I) in which R⁷ denotes an alkyl group having 1 to 6 C atoms, in particular denotes an alkyl group having 1 to 2 C atoms, and the groups R⁵ and R⁶ and R⁸ stand for hydrogen.

In the compounds of the formula (I), the group R preferably stands for linear or branched alkylene having 1 to 10 C atoms, in particular having 1 to 4 C atoms. One or more non-adjacent C atoms may preferably be replaced by O or S, preferably O.

In the preferred compounds of the formula (I), r is preferably equal to 0.

Preference is furthermore given to compounds of the formula (I) in which B is a single bond, —O—, —S—, —C(O)—O— or —O—C(O)—, in particular a single bond.

Particularly advantageous for use for increasing the efficacy of the antioxidants are compounds of the formula (I) in which one or more of the variables Rfⁱ, Yⁱ, Zⁱ, Lⁱ, R¹ to R⁸, ci, c′i, di, ni, mi, R, r and B have the preferred meanings, in particular compounds in which the said variables have the particularly preferred meanings. Particularly advantageous for use for increasing the efficacy of the antioxidants are compounds of the formula (I) in which all said variables have the preferred meanings, in particular the particularly preferred meanings.

In the compounds of the formula (I), X is a hydrophilic group, preferably an anionic, cationic, nonionic or amphoteric group.

A preferred anionic group X can be selected from —COO⁻, —SO₃⁻, —OSO₃⁻, —PO₃²⁻, —OPO₃²⁻, —(OCH₂CH₂)ₛ—O—(CH₂)ₜ—COO⁻, —(OCH₂CH₂)ₛ—O—(CH₂)ₜ—SO₃²⁻, —(OCH₂CH₂)ₛ—O—(CH₂)ₜ—OSO₃⁻, —(OCH₂CH₂)ₛ—O—(CH₂)ₜ—PO₃²⁻, —(OCH₂CH₂)ₛ—O—(CH₂)ₜ—OPO₃²⁻ or from the formulae (A*) to (C*),

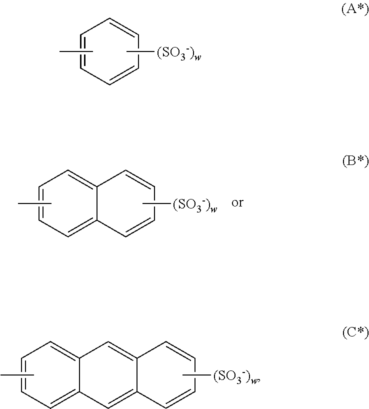

where s stands for an integer from the range from 1 to 1000, t stands for an integer selected from 1, 2, 3 or 4 and w stands for an integer selected from 1, 2 or 3.

The preferred anionic groups here include, in particular, —COO⁻, —SO₃⁻, —OSO₃⁻, —PO₃²⁻, —OPO₃²⁻, the sub-formula (A*), and —(OCH₂CH₂)ₛ—O—(CH₂)ₜ—COO⁻, —(OCH₂CH₂)ₛ—O—(CH₂)ₜ—SO₃⁻ and —(OCH₂CH₂)ₛ—O—(CH₂)ₜ—OSO₃⁻, where each individual one of these groups may be preferred per se.

The very particularly preferred anionic groups here include —SO₃⁻, —OSO₃⁻, —PO₃²⁻ or OPO₃²⁻. Especial preference is given to a sulfonate group —SO₃⁻.

The preferred counterion for anionic groups X is a monovalent cation, in particular H⁺, an alkali metal cation or N(R*)₄⁺, where R* stands for H or a straight-chain or branched alkyl group having 1 to 6 C atoms and all R* may be identical or different. Na⁺, K⁺ and NH₄⁺ are especially preferred, Na⁺ is particularly preferred.

A preferred cationic group X is selected from —[NR¹*R²*R³*]⁺Z⁻, —[PR¹*R²*R³*]⁺Z⁻,

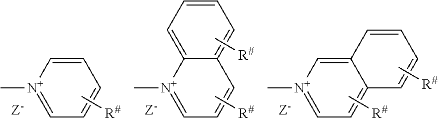

where R^{#} stands for H or a straight-chain or branched alkyl group having 1 to 4 C atoms in any desired position,

Z⁻ stands for Cl⁻, Br⁻, I⁻, CH₃SO₃⁻, CF₃SO₃⁻, CH₃PhSO₃⁻, PhSO₃⁻,

R¹, R² and R³ each stand, independently of one another, for H, a straight-chain or branched alkyl group having 1 to 30 C atoms, Ar or —CH₂Ar and

Ar stands for an unsubstituted or mono- or polysubstituted aromatic ring or condensed ring systems having 6 to 30 C atoms, in which, in addition, one or two CH groups may be replaced by N.

The abbreviation Ph in the definition of Z⁻ denotes phenyl.

Aryl having 6 to 30 C atoms denotes an aryl group having 6 to 30 C atoms and is an aromatic group having a common aromatic electron system having 6 to 30 C atoms, optionally mono- or polysubstituted by R¹*, OR¹, N(R¹)₂, CN, NO₂ or halogen. An aryl group having 6 to 34 C atoms, preferably having 6 to 24 C atoms, is preferably 1-, 2-, 3-, 4-, 5- or 6-phenyl, 1-, 2-, 3-, 4-, 6-, 7- or 8-naphthyl, 1-, 2-, 3-, 4-, 6-, 7- or 8-phenanthrenyl, 1-, 2-, 3-, 4-, 5-, 6-, 7-, 8-, 9- or 10-anthracenyl, 1-, 2-, 3-, 4-, 5-, 6-, 7-, 8-, 9-, 10-, 11- or 12-tetracenyl, 1-, 2-, 3-, 4-, 5-, 6-, 7-, 8-, 9-, 10-, 11- or 12-benzo[a]anthracenyl, 1-, 2-, 3-, 4-, 5-, 6-, 7-, 8-, 9-, 10-, 11-, 12-, 13- or 15-pentacenyl, 1-, 2-, 3-, 4-, 5-, 6-, 7-, 8-, 9-, 10-, 11- or 12-chrysenyl, 1-, 2-, 3-, 4-, 5-, 6-, 7-, 8-, 9- or 10-pyrenyl, 1-, 2-, 3-, 4-, 5-, 6-, 7-, 8-, 9-, 10-, 11- or 12-benzo[a]-pyrenyl, 1-, 2-, 3-, 4-, 5-, 6-, 7- or 8-azulenyl, 1-, 2-, 3-, 4-, 5-, 6-, 7-, 8-, 9- or 10-fluoranthenyl, 1-, 2-, 3-, 4-, 5-, 6-, 7-, 8-, 9-, 10-, 11- or 12-perylenyl, 1-, 2-, 3-, 4-, 5-, 6- or 7-indenyl or 1-, 2-, 3-, 4-, 5-, 6-, 7-, 8- or 9-fluorenyl, each of which is unsubstituted or substituted by R^{#}, OR^{#}, N(R^{#})₂, CN or halogen.

The preferred cationic groups here include, in particular, —[NR¹*R²*R³*]⁺Z⁻ and

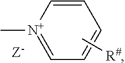

where each individual one of these groups may be preferred per se.

A preferred nonionic group for X can be selected from a linear or branched alkyl group, preferably having 1 to 20 C atoms, where one or more non-adjacent C atoms may be replaced by O, S, and/or N, —OH, —SH, —O-(glycoside)ₒ, —S-(glycoside)ₒ, —OCH₂—CHOH—CH₂—OH, —OCH₂Ar(—NCO)ₚ, —OAr(—NCO)ₚ, —CR″═CH₂, —OCOCR″═CH₂, amine oxide,

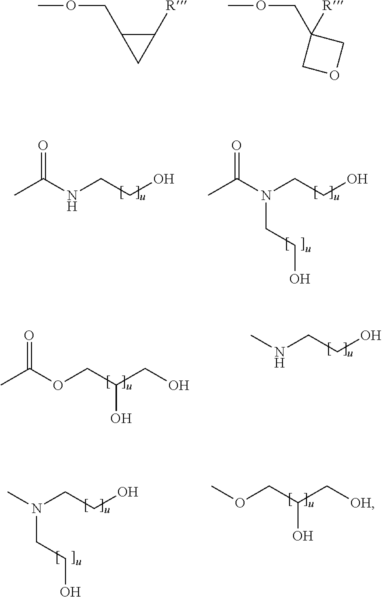

where

u stands for an integer from the range from 1 to 6, preferably for 1 to 4,

o stands for an integer from the range from 1 to 10,

p stands for 1 or 2,

R′″ stands, independently of one another, for C₁₋₃₀-alkyl, Ar or —CH₂Ar, preferably for C₁₋₂₀-alkyl, and

Ar stands for an unsubstituted, mono- or polysubstituted aromatic ring or condensed ring systems having 6 to 30 C atoms in which, in addition, one or two CH groups may be replaced by C═O,

glycoside stands for an etherified carbohydrate, preferably for a mono- di-, tri- or oligoglucoside,

and R″ stands for H or methyl.

The preferred nonionic groups here include, in particular, linear or branched alkyl, preferably having 1 to 20 C atoms, where one or more non-adjacent C atoms may be replaced by O, S and/or N, —OH, —OCOCR″═CH₂ and —O-(glycoside)ₒ.

If X is selected with the meaning linear or branched alkyl, where one or more non-adjacent C atoms have been replaced by O, S and/or N, this can be described synonymously by the formula R**—(B**-A**)_{m**}-,

where

R** stands for H or C₁₋₄-alkyl, in particular for H or CH₃, A** stands for a linear or branched alkylene, preferably having 1 to 10 carbon atoms, in particular having 1 to 4 carbon atoms, B** stands for O or S, preferably for O, and m** stands for an integer from 1 to 100, particularly preferably for an integer from 1 to 30.

The nonionic group X is particularly preferably the R**—(O—CH₂CHR^{a})_{m**}— group, where m** stands for an integer from 1 to 100, preferably for an integer from 1 to 30, in particular for an integer from 1 to 15, and R** and R^{a} each stand, independently of one another, for H or C₁₋₄-alkyl, in particular each stand, independently of one another, for H or CH₃. R**—(O—CH₂CHR^{a})_{m**}— is especially preferably a polyethylene or polypropylene glycol unit.

A preferred amphoteric group can be selected from the functional groups of the acetyldiamines, the N-alkylamino acids, the betaines or corresponding derivatives, in particular selected from:

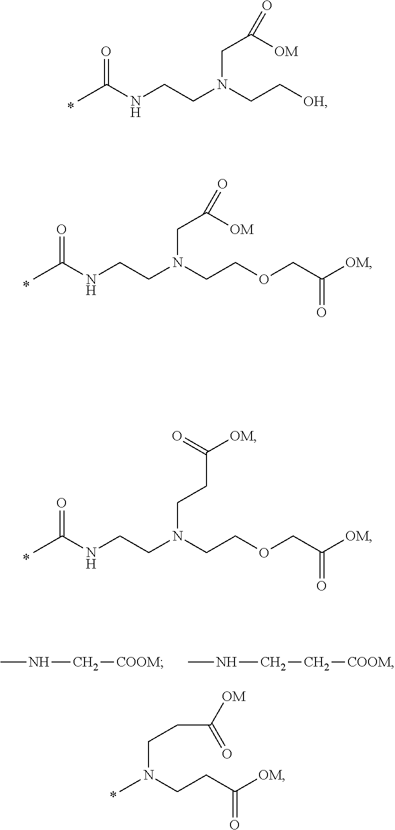

where M denotes a counterion, as described above or preferably described

—[(C(═O)—NH—(CH₂)₍₁₋₈₎]_{(0 or 1)}—N⁺R¹R²—CH₂—COO⁻, where R¹ and R² each stand, independently of one another, for a C1-8-alkyl radical, preferably methyl or ethyl

—C(═O)—NH—(CH₂)₁₋₃—N⁺R¹R²—CH₂—CH(OH)—CH₂-(O)_{(0 or 1)}—(S or P)O₃⁻, where R¹ and R² each stand, independently of one another, for a C1-8-alkyl radical, preferably methyl or ethyl.

Particularly preferred compounds of the formula (I) are those which contain one of the preferred anionic groups X, the preferred nonionic groups or the preferred zwitterionic groups as hydrophilic group X.

Especial preference is given to compounds of the formula (I) which contain the groups —SO₃⁻, —OSO₃⁻, —PO₃²⁻ or OPO₃²⁻, polyethylene glycol or polypropylene glycol, betaines, or sulfobetaines. Very particular preference is given to compounds of the formula (I) which contain the —SO₃⁻ group as hydrophilic group X. Preferred counterions here are Na⁺, K⁺ and NH₄⁺, in particular Na⁺.

Particularly advantageously used in accordance with the invention for increasing the efficacy of antioxidants are compounds of the formula (I) in which X is an anionic group, in particular —SO₃⁻, and one or more of the variables Rfⁱ, Yⁱ, Zⁱ, Lⁱ, R¹ to R⁸, ci, c′i, di, ni, mi, R, r and B have the preferred meanings described, in particular compounds in which the said variables have the particularly preferred meanings. Preferred compounds here are, in particular, compounds in which all variables have the preferred meanings, especially the particularly preferred meanings.

In a particularly preferred group of compounds of the formula (I), R¹ and R² stand for hydrogen and A¹ and A² stand for a —Zⁱ(CR³R⁴)ₘᵢRfⁱ group. These compounds are represented by formula (II). Particular preference is given to compounds of the formula (II) where Y¹, Y², Z¹ and Z² are equal to O.

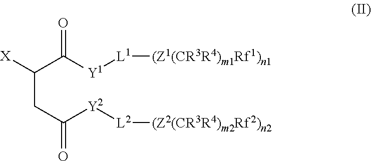

In another preferred group of compounds of the formula (I), R¹ stands for H, R² stands for —CH₂—COY³-L³-(A³)ₙ₃ and A¹, A² and A³ stand for a —Zⁱ(CR³R⁴)ₘᵢRfⁱ group. These compounds are represented by formula (III). Particular preference is given to compounds of the formula (III) where Y¹, Y², Z¹ and Z² are equal to O.

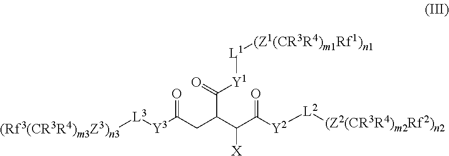

In a further preferred group of compounds of the formula (I), R¹ stands for —CH₂—COY³-L³-(A³)ₙ₃, R² stands for hydrogen and A¹, A² and A³ stand for a —Zⁱ(CR³R⁴)ₘᵢRfⁱ group. These compounds are represented by formula (IV). Particular preference is given to compounds of the formula (IV) where Y¹, Y², Y³, Z¹, Z² and Z³ are equal to O.

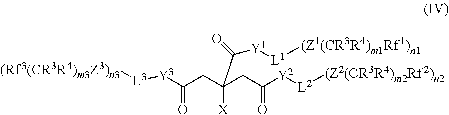

Particularly preferred compounds of the formula (I) are compounds of the formulae (II), (III) and (IV) in which X is an anionic group. Especial preference is given to compounds of the formulae (II), (III) and (IV) which contain the groups —SO₃⁻, —OSO₃⁻, —PO₃²⁻ or OPO₃²⁻, in particular —SO₃⁻. Preferred counterions here are Na⁺, K⁺ and NH₄⁺, in particular Na⁺.

In the formulae (II), (III) and (IV), L¹, L² and L³ have the general and preferred meanings given for the formula (I). Especial preference is given to compounds of the formulae (II), (III) and (IV) in which all Lⁱ are identical.

In addition, preference is given to compounds of the formulae (II), (III) and (IV) containing perfluorinated groups Rfⁱ having 1 to 4 C atoms. Rf¹, Rf² and Rf³ preferably have the same meaning. Preferred examples of the group Rfⁱ are pentafluoroethyl, n-heptafluoropropyl, n-nonafluorobutyl and —CH(CF₃)₂.

The invention accordingly furthermore relates to the use, as described above, characterised in that the compounds of the formula (I) used are compounds of the formulae (II), (III) and/or (IV),

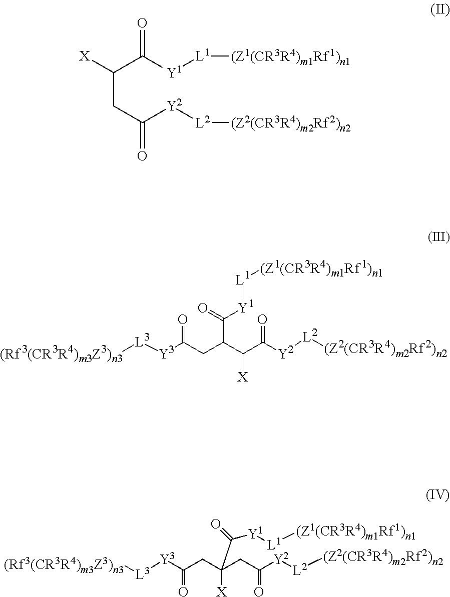

where

X stands for —SO₃⁻, —OSO₃⁻, —PO₃²⁻ or OPO₃²⁻, in particular for —SO₃⁻,

Y¹, Y², Y³, Z¹, Z² and Z³ stand for O,

Rf¹, Rf² and Rf³ stand for perfluorinated groups Rfⁱ having 1 to 4 C atoms,

n1, n2 and n3 stand for 1 or 2,

m1, m2 and m3 stand for 0, 1, 2, 3 or 4,

L¹, L² and L³ stand for a linear or branched alkylene having 2 to 6 C atoms and

R³ and R⁴ stand for hydrogen or an alkyl group having 1 to 3 C atoms.

In a preferred variant of the compounds of the formulae (II), (III) and (IV), R³ and R⁴ are identical. Preference is furthermore given to compounds in which R³ or R⁴ stand for hydrogen and m1, m2 and m3 stand for 0 or 1.

Preference is given to compounds in which all Rfⁱ, R³, R⁴, ni and mi in each case have the same meaning.

Particular preference is given to compounds of the formulae (II), (III) and (IV) in which all variables have the preferred meanings, in particular the particularly preferred meanings.

Use is particularly advantageously made of compounds of the formula (III) in which all variables have the preferred meanings, in particular the particularly preferred meanings.

Examples of compounds of the formula (I) or of the formula (III) whose use is particularly advantageous are compounds of the formulae (III-1) to (III-6):

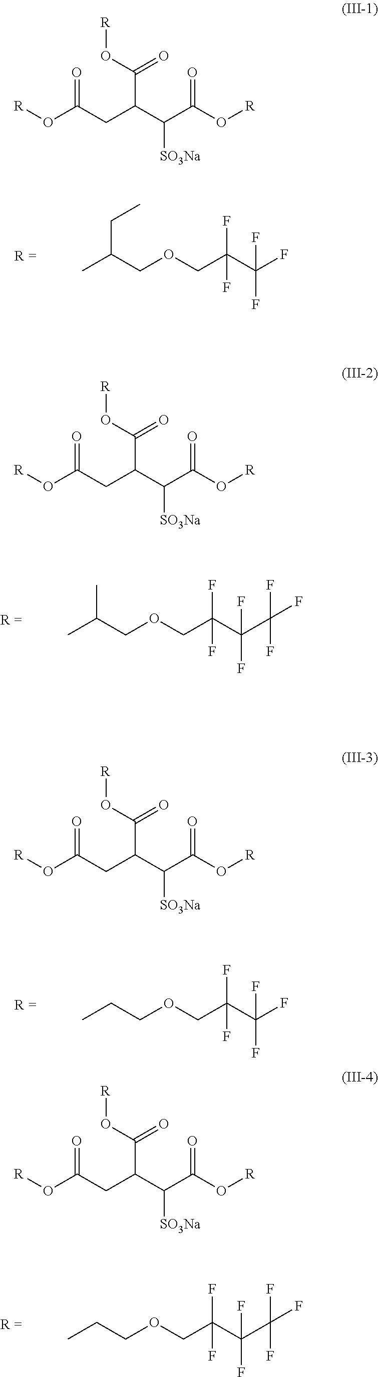

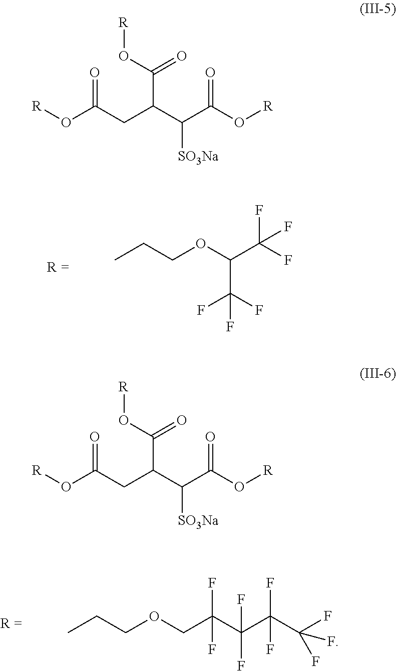

Examples of compounds of the formula (I) whose use is very particularly advantageous are compounds of the formulae (III-1) and (III-2), as described above.

The compounds of the formulae (I), (II), (III) and (IV) may also be in the form of isomer mixtures (constitutional and/or configurational isomer mixtures). In particular, diastereomer and/or enantiomer mixtures are possible. The formulae indicated encompass all these forms.

The compounds of the formula (I), as described above or described as preferred, in which Y¹ and Y² denote O can preferably be prepared by esterification of maleic acid and aconitic acid or anhydrides or acid chlorides thereof using one or more alcohols of the formula (V)

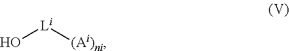

and subsequent addition onto the double bond in order to introduce the X—(R)ᵣ—B group, as described above. The compounds of the formula (I) can also preferably be prepared by esterification of hydroxy-succinic acid and citric acid using one or more alcohols of the formula (V) and subsequent functionalisation of the hydroxyl groups in order to introduce the X—(R)ᵣ—B group.

Lⁱ and Aⁱ in the formula (V) have the meaning described for L¹, L² and L³ and A¹, A² and A³ respectively in formula (I), in particular also the preferred meanings. The alcohols of the formula (V) may contain one or more Rfi groups, as described above.

The alcohols used are commercially available and/or their preparation is familiar to the person skilled in the art (for example DE 10 2009 030 846 A1; Heilmann et al. J. Fluorine Chem. 1992, 59, 387; Janulis et al. U.S. Pat. No. 5,157,159 (1992); Carbohydrate Research 1991, 219, 33).

The synthesis of the succinates or tricarballylates of the formula (I) is preferably carried out in a two-step synthesis via the corresponding maleates or hydroxysuccinates or the corresponding aconitic or citric acid esters. These syntheses are described in WO 2010/149262, in particular in examples 1, 5 to 9, WO 2011/082770 and WO 2012/084118. The disclosures in the references cited hereby expressly also belong to the disclosure content of the present application.

As described above, the compounds of the formula (I), (II), (III) or (IV) and the compounds described as preferred cause a boost effect of antioxidants.

Examples of antioxidants whose efficacy is enhanced are amino acids, imidazoles, peptides, carotenoids, carotenes, chlorogenic acid and derivatives thereof, lipoic acid and derivatives thereof, aurothioglucose, thiols, thiodipropionic acid and derivatives thereof, sulfoximine compounds, (metal) chelators, unsaturated fatty acids and derivatives thereof, vitamin C and derivatives, tocopherols and derivatives, vitamin A and derivatives, rutinic acid and derivatives thereof, flavonoids and derivatives thereof, ferulic acid, furfurylideneglucitol, carnosine, butylhydroxytoluene, butylhydroxyanisole, nordohydroguaiaretic acid, trihydroxybutyrophenone, uric acid and derivatives thereof, mannose and derivatives thereof, stilbenes and derivatives thereof, derivatives of benzylidene-2,4-alkanedione, derivatives of benzylidenemalonic acid or derivatives of benzylmalonic acid.

Accordingly, the invention furthermore relates to the use of compounds of the formula (I), (II), (III), (IV) or the preferred compounds, as described above, where the efficacy of antioxidants from the group amino acids, imidazoles, peptides, carotenoids, carotenes, chlorogenic acid and derivatives thereof, lipoic acid and derivatives thereof, aurothioglucose, thiols, thiodipropionic acid and derivatives thereof, sulfoximine compounds, (metal) chelators, unsaturated fatty acids and derivatives thereof, vitamin C and derivatives, tocopherols and derivatives, vitamin A and derivatives, rutinic acid and derivatives thereof, flavonoids and derivatives thereof, ferulic acid, furfurylideneglucitol, carnosine, butylhydroxytoluene, butylhydroxyanisole, nordohydroguaiaretic acid, trihydroxybutyrophenone, uric acid and derivatives thereof, mannose and derivatives thereof, stilbenes and derivatives thereof, derivatives of benzylidene-2,4-pentanedione, derivatives of benzylidenemalonic acid or derivatives of benzylmalonic acid is enhanced.

Suitable amino acids are, for example, glycine, histidine, tyrosine, tryptophan, thiotaine.

A suitable imidazole is, for example, urocanic acid and derivatives thereof.

Suitable derivatives are, for example, salts with cosmetically tolerated cations, esters and ethers.

Suitable peptides are, for example, D,L-carnosine, D-carnosine, L-carnosine and derivatives thereof, for example anserine.

Suitable carotenes are, for example, α-carotene, β-carotene, lycopene.

A suitable derivative of lipoic acid is dihydrolipoic acid.

Suitable thiols are, for example, thioredoxin, glutathione, cysteine, cystine, cystamine and glycosyl, N-acetyl, methyl, ethyl, propyl, amyl, butyl, lauryl, palmitoyl, oleyl, α-linoleyl, cholesteryl and glyceryl esters thereof and salts thereof.

Suitable derivatives of thiodipropionic acid are, for example, dilauryl thiodipropionate, distearyl thiodipropionate, ethers thereof, peptides, lipids, nucleotides, nucleosides and salts thereof.

Suitable suloximine compounds are, for example, buthionine sulfoximine, homocysteine sulfoxime, buthionine sulfone, penta-, hexa- and heptathionine sulfoximine.

Suitable (metal) chelators are α-hydroxyfatty acids, palmitic acid, phytic acid, lactoferrin, α-hydroxy acids, such as citric acid, lactic acid, malic acid, humic acid, bile acid, bile extracts, bilirubin, biliverdin, EDTA, EGTA or unsaturated fatty acids.

Suitable derivatives of vitamin C are ascorbyl palmitate, magnesium ascorbyl phosphate or ascorbyl acetate.

A suitable derivative of tocopherol is tocopherol acetate.

A suitable derivative of vitamin A is vitamin A palmitate.

The term “flavonoid” encompasses, in particular, compounds which, owing to their basic structure, can be assigned to the following groups:
chalcones
chromones
aurones
flavanones
flavan-3-ols (catechols)
flavones
isoflavones
flavan-3,4-diols (leukoanthocyanidines)
flavonols (3-hydroxyflavan-4-one)
flavanonols

The following flavonoids should be mentioned by way of example: 5-hydroxy-7,4″-dimethoxyflavone 8-sulfate, 7,8-dihydroxyflavone, luteolin (flavones); catechol, epicatechol, epigallocatechol gallate (EGCG, TEAVIGO® DSM) (flavan-3-ols or flavan-3-ol derivatives); kaempferol (flavonol); taxifolin (flavanonol), the isoflavones from the group isoflavone, daidzein, genistein, prunetin, biochanin A, orobol, santal or pratensein, as well as naringenin (flavanone), and glyco-sides of naringenin, for example naringin 7-neohesperidoside.

Preferred flavonoids are derived from the following groups:
flavonols
flavonol o-glycosides
flavonol o-glycoside-containing extracts

The preferred flavonoids also include aglycones (sugar-free structures) and aglycone conjugates. Possible aglycone conjugates are hydroxyl derivatives, where all or some of the hydroxyl groups are alkylated, methylated, glycylated, sulfated or esterified. Besides hydroxyl derivatives, C derivatives are also suitable as aglycone conjugates.

For the group of the flavonols, particular preference is given to the aglycone quercetin.

In the group of the flavonol o-glycosides, the flavonol 3-glycosides, such as rutin, α-glucosylrutin, tiliroside, isoquercetin, rutin sulfate, trishydroxyethylrutin (troxerutin), and sulfates and phosphates thereof are particularly preferred.

Flavonol 7- and 8-glycosides can also be used.

The term “rutin sulfate” encompasses mono-, di-, tri-, tetra- or poly-sulfates of rutin and mixtures of these rutin sulfates. The term “troxerutin” encompasses mono-, di-, tri-, tetra- or polyethoxylates of rutin and mixtures of these rutin ethoxylates.

For the group of the flavonol- or flavonol o-glycoside-containing extracts, the active compound combinations emblica, liquorice and/or horse chestnut extract are preferred. Emblica is obtained from the fruit of the deciduous tree Phyllanthus emblica (also Emblica officinalis), for example in India, China, Pakistan or Nepal. The principal ingredients of emblica are the low-molecular-weight tannic acids emblicanin A and B, which bind the iron occurring in the skin in the form of complexes. Preferred emblica solutions are commercially available, for example as EMBLICA® (MERCK) or CAPROS®. All emblica mixtures, alone or in combination, are in principle suitable as antioxidant.

The liquorice extract contains the flavonoid glabridin (a stearyl glycyrhetinate) and/or licochalcone A.

The horse chestnut extract contains, for example, esculin and other flavonol and/or flavonol glycoside constituents.

Plant substance mixtures of this type can be obtained in a manner familiar to the person skilled in the art, for example by pressing or extraction of the corresponding plants.

In a preferred embodiment of the invention, the efficacy of the flavonoids selected from the group quercetin, rutin, rutin sulfate, α-glucosylrutin, tiliroside, troxerutin and/or isoquercetin is increased.

In a further preferred embodiment of the invention, the efficacy of derivatives of benzylidene-2,4-alkanedione, derivatives of benzylidenemalonic acid or derivatives of benzylmalonic acid, which are described by the formulae (A) and (B), is increased.

The invention therefore furthermore relates to the use of compounds of the formula (I), (II), (III), (IV) or preferred compounds, as described above, for increasing the efficacy of compounds of the formula (A) and/or (B),

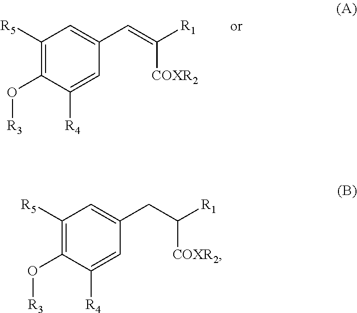

in which

R₁ denotes —C(O)R₆, —CO₂R₆ or —C(O)N(R₇)₂,

R₂ denotes linear or branched alkyl having 1 to 30 C atoms,

X O, NH or a bond,

R₃ denotes H or linear or branched alkyl having 1 to 20 C atoms,

R₄ and R₅ each, independently of one another, denote H, linear or branched alkyl having 1 to 12 C atoms or linear or branched alkoxy having 1 to 12 C atoms,

R₆ denotes linear or branched alkyl having 1 to 30 C atoms and R₇ in each case, independently of one another, denotes H or linear or branched alkyl having 1 to 8 C atoms.

Preferred derivatives of benzylidene-2,4-alkanedione are compounds of the formula (A) in which R₁ denotes —C(O)R₆ and X denotes a bond, corresponding to the formula (A-1),

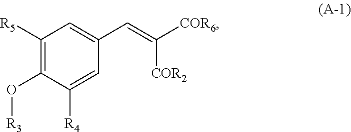

where R₂, R₃, R₄, R₅, R₆ and R₇ have a meaning given above for formula (A).

In compounds of the formula (A-1), R₃ is preferably a linear or branched alkyl group having 1 to 4 C atoms.

In compounds of the formula (A-1), R₄ and R₅ are preferably identical and are a linear or branched alkoxy group having 1 to 4 C atoms.

In compounds of the formula (A-1), R₆ and R₂ are preferably identical and are a linear or branched alkyl group having 1 to 4 C atoms.

Particularly preferred compounds of the formula (A-1) are compounds in which R₃ is preferably a linear or branched alkyl group having 1 to 4 C atoms, R₄ and R₅ are preferably identical and are a linear or branched alkoxy group having 1 to 4 C atoms and R₆ and R₂ are preferably identical and are a linear or branched alkyl group having 1 to 4 C atoms.

Preferred compounds of the formula (A-)1 are, for example, 3-(3,4,5-trimethoxybenzylidene)-2,4-pentanedione, 3-(3,4,5-triethoxybenzylidene)-2,4-pentanedione, 3-(3,4,5-tripropoxybenzylidene)-2,4-pentanedione, 3-(3,4,5-tributoxybenzylidene)-2,4-pentanedione, 3-(3,4,5-trimethoxybenzylidene)-2,4-heptanedione, 3-(3,4,5-triethoxybenzylidene)-2,4-heptanedione, 3-(3,4,5-tripropoxybenzylidene)-2,4-heptanedione, 3-(3,4,5-tributoxybenzylidene)-2,4-heptanedione, 3-(3,4,5-trimethoxybenzylidene)-2,4-nonanedione, 3-(3,4,5-triethoxybenzylidene)-2,4-nonanedione, 3-(3,4,5-tripropoxybenzylidene)-2,4-nonanedione, 3-(3,4,5-tributoxybenzylidene)-2,4-nonanedione. A particularly preferred compound of the formula (A-1) is 3-(3,4,5-trimethoxybenzylidene)-2,4-pentanedione. This compound is commercially available under the trade name Synoxyl® HSS from Sytheon Ltd.

Preferred derivatives of benzylidenemalonic acid are compounds of the formula (A) in which R₁ denotes —CO₂R₆ and X denotes O, corresponding to the formula (A-2),

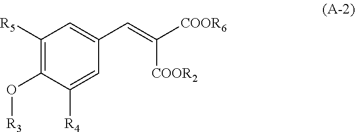

where R₂, R₃, R₄, R₅, R₆ and R₇ have a meaning given above for formula (A).

In compounds of the formula (A-2), R₃ is preferably H or a linear or branched alkyl group having 1 to 4 C atoms.

In compounds of the formula (A-2), R₃ is particularly preferably H.

In compounds of the formula (A-2), R₄ is preferably H or a linear or branched alkoxy group having 1 to 4 C atoms.

In compounds of the formula (A-2), R₄ is particularly preferably a linear or branched alkoxy group having 1 to 4 C atoms.

In compounds of the formula (A-2), R₅ is preferably H or a linear or branched alkoxy group having 1 to 4 C atoms.

In compounds of the formula (A-2), R₅ is particularly preferably a linear or branched alkoxy group having 1 to 4 C atoms.

In compounds of the formula (A-2), R₆ and R₂ are preferably identical and are a linear or branched alkyl group having 1 to 12 C atoms.

Particularly preferred compounds of the formula (A-2) are compounds in which R₃ is preferably H or a linear or branched alkyl group having 1 to 4 C atoms, R₄ is preferably H or a linear or branched alkoxy group having 1 to 4 C atoms, R₅ is preferably H or a linear or branched alkoxy group having 1 to 4 C atoms and R₆ and R₂ are preferably identical and are a linear or branched alkyl group having 1 to 12 C atoms.

Preferred compounds of the formula (A-2) are bis(2-ethylhexyl) 2-(4-hydroxy-3,5-dimethoxybenzylidene)malonate, bis(2-ethylhexyl) 2-(4-hydroxy-3,5-diethoxybenzylidene)malonate, bis(2-ethylhexyl) 2-(4-hydroxy-3,5-dipropoxybenzylidene)malonate, bismethyl 2-(4-hydroxy-3,5-dimethoxybenzylidene)malonate, bismethyl 2-(4-hydroxy-3,5-diethoxybenzylidene)malonate, bismethyl 2-(4-hydroxy-3,5-dipropoxybenzylidene)malonate, bisisopropyl 2-(4-hydroxy-3,5-dimethoxybenzylidene)malonate, bisisopropyl 2-(4-hydroxy-3,5-diethoxybenzylidene)malonate, bisisopropyl 2-(4-hydroxy-3,5-dipropoxybenzylidene)malonate, bisisopropyl 2-(4-hydroxy-3-methoxybenzylidene)malonate, bisisopropyl 2-(4-hydroxy-3-ethoxy-benzylidene)malonate, bisisopropyl 2-(4-hydroxy-3-propoxy-benzylidene)malonate, bismethyl 2-(4-hydroxy-3-methoxy-benzylidene)malonate, bismethyl 2-(4-hydroxy-3-ethoxybenzylidene)-malonate, bismethyl 2-(4-hydroxy-3-propoxybenzylidene)malonate, bis(2-ethylhexyl) 2-(4-hydroxy-3-methoxybenzylidene)malonate, bis(2-ethylhexyl) 2-(4-hydroxy-3-ethoxybenzylidene)malonate, bis(2-ethylhexyl) 2-(4-hydroxy-3-propoxybenzylidene)malonate, bis(2-ethylhexyl) 2-(4-methoxybenzylidene)malonate, bis(2-ethylhexyl) 2-(4-ethoxybenzylidene)malonate, bis(2-ethylhexyl) 2-(4-propoxybenzylidene)malonate, bismethyl 2-(4-methoxybenzylidene)malonate, bismethyl 2-(4-ethoxybenzylidene)malonate, bismethyl 2-(4-propoxybenzylidene)malonate, bisisopropyl 2-(4-methoxybenzylidene)-malonate, bisisopropyl 2-(4-ethoxybenzylidene)malonate, bisisopropyl 2-(4-propoxybenzylidene)malonate.

Particularly preferred compounds of the formula (A-2) are bis(2-ethylhexyl) 2-(4-hydroxy-3,5-dimethoxybenzylidene)malonate, bisisopropyl 2-(4-hydroxy-3-methoxybenzylidene)malonate and bis(2-ethylhexyl) 2-(4-methoxybenzylidene)malonate.

A very particularly preferred compound of the formula (A-2) is bis(2-ethylhexyl) 2-(4-hydroxy-3,5-dimethoxybenzylidene)malonate, which is marketed as an active constituent of the proprietary product Oxynex® ST Liquid from Merck.

Preferred derivatives of benzylmalonic acid are compounds of the formula (B) in which R₁ denotes —CO₂R₆ and X denotes O, corresponding to the formula (B-1),

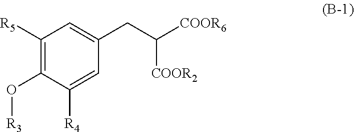

where R₂, R₃, R₄, R₅, R₆ and R₇ have a meaning given above for formula (B).

In compounds of the formula (B-1), R₃ is preferably H or a linear or branched alkyl group having 1 to 4 C atoms.

In compounds of the formula (B-1), R₃ is particularly preferably H.

In compounds of the formula (B-1), R₄ is preferably H or a linear or branched alkoxy group having 1 to 4 C atoms.

In compounds of the formula (B-1), R₄ is particularly preferably a linear or branched alkoxy group having 1 to 4 C atoms.

In compounds of the formula (B-1), R₅ is preferably H or a linear or branched alkoxy group having 1 to 4 C atoms.

In compounds of the formula (B-1), R₅ is particularly preferably a linear or branched alkoxy group having 1 to 4 C atoms.

In compounds of the formula (B-1), R₆ and R₂ are preferably identical and are a linear or branched alkyl group having 1 to 12 C atoms.

Particularly preferred compounds of the formula (B-1) are compounds in which R₃ is preferably H or a linear or branched alkyl group having 1 to 4 C atoms, R₄ is preferably H or a linear or branched alkoxy group having 1 to 4 C atoms, R₅ is preferably H or a linear or branched alkoxy group having 1 to 4 C atoms and R₆ and R₂ are preferably identical and are a linear or branched alkyl group having 1 to 12 C atoms.

Preferred compounds of the formula (B-1) are bis(2-ethylhexyl) 2-(4-hydroxy-3,5-dimethoxybenzyl)malonate, bis(2-ethylhexyl) 2-(4-hydroxy-3,5-diethoxybenzyl)malonate, bis(2-ethylhexyl) 2-(4-hydroxy-3,5-dipropoxybenzyl)malonate, bismethyl 2-(4-hydroxy-3,5-dimethoxybenzyl)malonate, bismethyl 2-(4-hydroxy-3,5-diethoxybenzyl)malonate, bismethyl 2-(4-hydroxy-3,5-dipropoxybenzyl)-malonate, bisisopropyl 2-(4-hydroxy-3,5-dimethoxybenzyl)malonate, bisisopropyl 2-(4-hydroxy-3,5-diethoxybenzyl)malonate, bisisopropyl 2-(4-hydroxy-3,5-dipropoxybenzyl)malonate, bisisopropyl 2-(4-hydroxy-3-methoxybenzyl)malonate, bisisopropyl 2-(4-hydroxy-3-ethoxybenzyl)malonate, bisisopropyl 2-(4-hydroxy-3-propoxybenzyl)-malonate, bismethyl 2-(4-hydroxy-3-methoxybenzyl)malonate, bismethyl 2-(4-hydroxy-3-ethoxybenzyl)malonate, bismethyl 2-(4-hydroxy-3-propoxybenzyl)malonate, bis(2-ethylhexyl) 2-(4-hydroxy-3-methoxybenzyl)malonate, bis(2-ethylhexyl) 2-(4-hydroxy-3-ethoxybenzyl)malonate, bis(2-ethylhexyl) 2-(4-hydroxy-3-propoxybenzyl)-malonate, bis(2-ethylhexyl) 2-(4-methoxybenzyl)malonate, bis(2-ethylhexyl) 2-(4-ethoxybenzyl)malonate, bis(2-ethylhexyl) 2-(4-propoxybenzyl)malonate, bismethyl 2-(4-methoxybenzyl)malonate, bismethyl 2-(4-ethoxybenzyl)malonate, bismethyl 2-(4-propoxybenzyl)malonate, bisisopropyl 2-(4-methoxybenzyl)malonate, bisisopropyl 2-(4-ethoxybenzyl)malonate, bisisopropyl 2-(4-propoxybenzyl)malonate.

A very particularly preferred compound of the formula (B-1) is bis(2-ethylhexyl) 2-(4-hydroxy-3,5-dimethoxybenzyl)malonate, which is marketed by Merck under the proprietary name RonaCare® AP.

The compounds of the formula (A), (A-1), (A-2), (B) and (B-1), as described above or described as preferred, are in accordance with the invention enhanced in their action in a particularly preferred manner.

Accordingly, the invention furthermore relates to the use of compounds of the formula (I), (II), (III), (IV) or the preferred compounds, as described above, where the efficacy of the antioxidants from the group bis(2-ethylhexyl 2-(4-hydroxy-3,5-dimethoxybenzylidene)-malonate), bis(2-ethylhexyl) 2-(4-hydroxy-3,5-dimethoxybenzyl)-malonate, bisisopropyl 2-(4-hydroxy-3-methoxybenzylidene)-malonate, bisisopropyl 2-(4-hydroxy-3-methoxybenzyl)malonate, bis(2-ethylhexyl) 2-(4-methoxybenzylidene)malonate and/or 3-(3,4,5-trimethoxybenzylidene)-2,4-pentanedione is enhanced.

Accordingly, the invention furthermore relates to the use of compounds of the formula (I), (II), (III), (IV) or the preferred compounds, as described above, where the efficacy of the antioxidants from the group bis(2-ethylhexyl) 2-(4-hydroxy-3,5-dimethoxybenzylidene)-malonate and/or bis(2-ethylhexyl) 2-(4-hydroxy-3,5-dimethoxybenzyl)malonate is enhanced.

In order to work the invention, the compounds of the formula (I), (II), (III), (IV) or the preferred compounds, as described above, can be mixed with the antioxidants, as described above or preferably described. Mixtures of this type are then preferably incorporated into a cosmetic or dermatological preparation or a medicinal product.

Mixtures of this type may be a premix for a preparation, for example of pulverulent ingredients, which is used as such as a powder mixture with at least one suitable vehicle and optionally further ingredients for the preparation of the cosmetic or dermatological preparation or the medicinal product.

Conventional vehicles for a pulverulent mixture are sorbitol, lactose, talc, silicic acid, aluminium hydroxide, calcium silicate, polyamide powder or mixtures of these substances.

Suitable mixtures may likewise be a premix for a preparation, for example in the form of a paste or solution of the ingredients, which is used as such as a mixture with at least one suitable vehicle and optionally further ingredients for the preparation of the cosmetic or dermatological preparation or the medicinal product.

The mixture of the compounds of the formula (I), (II), (III), (IV) or the preferred compounds, as described above, with the antioxidants, as described above or preferably described, may include or comprise, essentially consist of or consist of the said requisite or optional constituents. All compounds or components which can be used in the preparations are either known and commercially available or can be synthesised by known processes.

The invention furthermore likewise relates to a mixture comprising a compound of the formula (I),

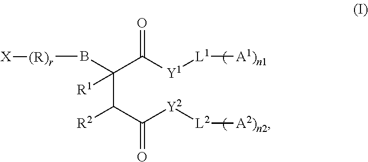

where

X is a hydrophilic group,

R is linear or branched alkylene having 1 to 12 C atoms, in which one or more non-adjacent C atoms may be replaced by O, S, and/or N,

r is 0 or 1,

B is a single bond, —O—, —NH—, —NR′—, —CH₂—, —C(O)—O—, —S—, —CH₂—O—, —O—C(O)—, —O—C(O)—O—, —N—C(O)—, —C(O)—N—, —O—C(O)—N—, —N—C(O)—N—, —Si(R′)₂—, —Si(R′)₂—O—, —O—SO₂— or —SO₂—O—,

where R′ is a linear or branched alkyl group having 1 to 12 C atoms,

R¹ and R², independently of one another, are hydrogen or —CH₂—COY³-L³-(A³)ₙ₃,

Y¹,Y² and Y³, independently of one another, are O, S or N,

L¹, L² and L³, independently of one another, are linear or branched alkylene having 1 to 12 C atoms, in which one or more non-adjacent C atoms may be replaced by O, S, and/or N,

A¹, A² and A³, independently of one another, are hydrogen or a group of the structure —Zⁱ(CR³R⁴)ₘᵢRfⁱ, where i denotes 1, 2 or 3,

Zⁱ is O, S or N and is bonded to a C atom of Lⁱ or is a single bond,

R³ and R⁴, independently of one another, are hydrogen or a straight-chain or branched alkyl group having 1 to 12 C atoms,

Rfⁱ is a fluorine-containing radical,

n1, n2 and n3, independently of one another, are 1, 2 or 3,

m1, m2 and m3, independently of one another, are 0, 1, 2, 3, 4 or 5, and the compounds of the formula (I) contain at least one Rfⁱ group, and at least one antioxidant, as described above or preferably described.

The invention furthermore likewise relates to a mixture comprising a compound of the formula (I)

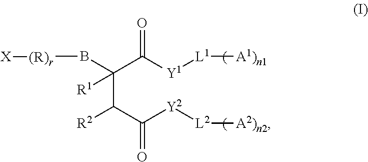

where

X is a hydrophilic group,

R is linear or branched alkylene having 1 to 12 C atoms, in which one or more non-adjacent C atoms may be replaced by O, S, and/or N,

r is 0 or 1,

B is a single bond, —O—, —NH—, —NR′—, —CH₂—, —C(O)—O—, —S—, —CH₂—O—, —O—C(O)—, —O—C(O)—O—, —N—C(O)—, —C(O)—N—, —O—C(O)—N—, —N—C(O)—N—, —Si(R′)₂—, —Si(R′)₂—O—, —O—SO₂— or —SO₂—O—,

where R′ is a linear or branched alkyl group having 1 to 12 C atoms,

R¹ and R², independently of one another, are hydrogen or —CH₂—COY³-L³-(A³)ₙ₃,

Y¹,Y² and Y³, independently of one another, are O, S or N,

L¹, L² and L³, independently of one another, are linear or branched alkylene having 1 to 12 C atoms, in which one or more non-adjacent C atoms may be replaced by O, S, and/or N,

A¹, A² and A³, independently of one another, are hydrogen or a group of the structure —Zⁱ(CR³R⁴)ₘᵢRfⁱ, where i denotes 1, 2 or 3,

Zⁱ is O, S or N and is bonded to a C atom of Lⁱ or is a single bond,

R³ and R⁴, independently of one another, are hydrogen or a straight-chain or branched alkyl group having 1 to 12 C atoms,

Rfⁱ is a fluorine-containing radical,

n1, n2 and n3, independently of one another, are 1, 2, or 3,

m1, m2 and m3, independently of one another, are 0, 1, 2, 3, 4 or 5, and the compounds of the formula (I) contain at least one Rfⁱ group, and one or more compounds of the formula (A) and/or (B)

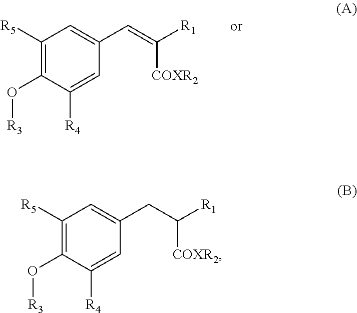

in which

R₁ denotes —C(O)R₆, —CO₂R₆ or —C(O)N(R₇)₂,

R₂ denotes linear or branched alkyl having 1 to 30 C atoms,

X O, NH or a bond,

R₃ denotes H or linear or branched alkyl having 1 to 20 C atoms,

R₄ and R₅ each, independently of one another, denote H, linear or branched alkyl having 1 to 12 C atoms or linear or branched alkoxy having 1 to 12 C atoms,

R₆ denotes linear or branched alkyl having 1 to 30 C atoms and

R₇ in each case, independently of one another, denotes H or linear or branched alkyl having 1 to 8 C atoms.

Preferred mixtures are mixtures of preferred compounds of the formula (I), (II), (III) or (IV) with preferred compounds of the formula (A), (A-1), (A-2), (B) or (B-1).

Particularly preferred mixtures are mixtures of preferred compounds of the formula (I), (II), (III) or (IV) with the compounds bis(2-ethylhexyl) 2-(4-hydroxy-3,5-dimethoxybenzylidene)malonate), bis(2-ethylhexyl) 2-(4-hydroxy-3,5-dimethoxybenzyl)malonate, bisisopropyl 2-(4-hydroxy-3-methoxybenzylidene)malonate, bisisopropyl 2-(4-hydroxy-3-methoxybenzyl)malonate, bis(2-ethylhexyl) 2-(4-methoxybenzylidene)malonate and/or 3-(3,4,5-trimethoxybenzylidene)-2,4-pentanedione.

The invention furthermore relates to mixtures comprising bis(2-ethylhexyl) 2-(4-hydroxy-3,5-dimethoxybenzylidene)malonate and/or bis(2-ethylhexyl) 2-(4-hydroxy-3,5-dimethoxybenzyl)malonate and the compound of the formula (III-1):

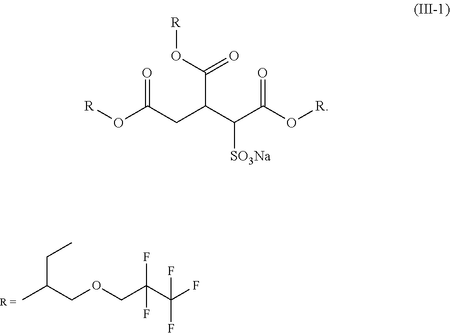

The mixtures may preferably comprise assistants, such as, for example, cosmetic oils (for example Caprylic/Capric Triglycerides, C12-15 Alkyl Benzoate, isopropyl myristate, Arylalkyl Benzoate, such as, for example, phenethyl benzoate (X-Tend 226) or oil components of the Cosmacol brand, such as Dimyristyl Tartrate, Tri C14-C15 Alkyl Citrate, C12-C13 Alkyl Lactate, Tridecyl Salicylate, C12-C13 Alkyl Octanoate, C12-C13 Alkyl Malate, C12-C13 Alkyl Citrate, C12-C13 Alkyl Tartrate), or polar-protic assistants (for example propylene glycol, butylene glycol, glycerin, isopropanol, ethanol) or so-called solubilisers (for example butylphthalimides, isopropylphthalimides, dimethylisosorbides). Very particularly preferred cosmetic oils are C12-C13 Alkyl Lactate, commercially available as Cosmacol ELI, and phenethyl benzoate, commercially available as X-Tend 226.

Preference is given to the use of the compound bis(2-ethylhexyl) 2-(4-hydroxy-3,5-dimethoxybenzylidene)malonate without a further assistant and a compound of the formula (I), (II), (III) or (IV), in particular with the compound of the formula (III-1), in the mixture. A preferred assistant is a mixture of caprylic acid triglyceride and capric acid triglyceride having the INCI name caprylic/capric triglyceride.

Alternatively, use is made of the compound bis(2-ethylhexyl) 2-(4-hydroxy-3,5-dimethoxybenzylidene)malonate together with a cosmetic assistant and a compound of the formula (I), (II), (III) or (IV), in particular with the compound of the formula (III-1), in the mixture. A preferred assistant is a mixture of caprylic acid triglyceride and capric acid triglyceride having the INCI name caprylic/capric triglyceride. An alternative assistant is 1-methoxy-2-propanol.

Preference is given to the use of the compound bis(2-ethylhexyl) 2-(4-hydroxy-3,5-dimethoxybenzylidene)malonate together with bis(2-ethylhexyl) 2-(4-hydroxy-3,5-dimethoxybenzyl)malonate and a compound of the formula (I), (II), (III) or (IV), in particular with the compound of the formula (III-1), in the mixture.

Preference is given to the use of the compound bis(2-ethylhexyl) 2-(4-hydroxy-3,5-dimethoxybenzyl)malonate without a further assistant and a compound of the formula (I), (II), (III) or (IV), in particular with the compound of the formula (III-1), in the mixture. The mixture is then preferably a solution.

The invention therefore furthermore relates to a mixture consisting of bis(2-ethylhexyl) 2-(4-hydroxy-3,5-dimethoxybenzyl)malonate and the compound of the formula (III-1):

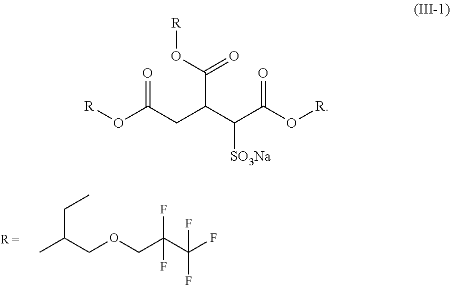

The invention furthermore likewise relates to a mixture consisting of bis(2-ethylhexyl) 2-(4-hydroxy-3,5-dimethoxybenzyl)malonate bis(2-ethylhexyl) 2-(4-hydroxy-3,5-dimethoxybenzyl)malonate and the compound of the formula (III-1):

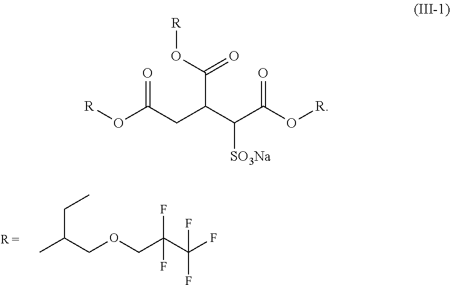

The invention furthermore likewise relates to a mixture consisting of bis(2-ethylhexyl) 2-(4-hydroxy-3,5-dimethoxybenzylidene)malonate and the compound of the formula (III-1):

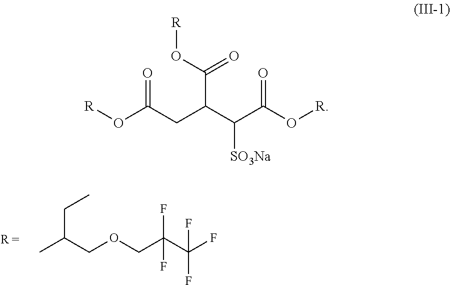

The invention furthermore likewise relates to a mixture consisting of bis(2-ethylhexyl) 2-(4-hydroxy-3,5-dimethoxybenzylidene)malonate, 1-methoxy-2-propanol and the compound of the formula (III-1):

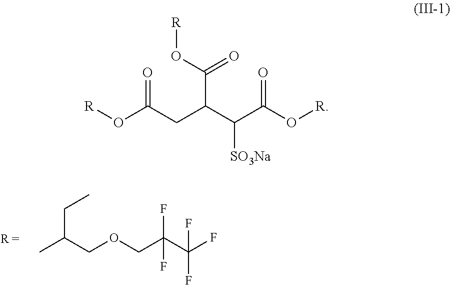

The mixture, as described above or preferably described, preferably comprises a compound of the formula (I) and an antioxidant in a weight ratio of 1:1 to 1:20, preferably in a weight ratio of 1:4 to 1:15, particularly preferably in a weight ratio of 1:9. If a plurality of compounds of the formula (I) or a plurality of antioxidants are employed together, the total amount is determinant for the weight ratio.

The invention furthermore likewise relates to preparations, in particular cosmetic or dermatological preparations, or medicinal products comprising at least one compound of the formula (I), as described above or described as preferred, at least one antioxidant, as described above or preferably described, or a mixture according to the invention, as described above or described as preferred.

The invention furthermore likewise relates to preparations comprising at least one compound of the formula (I), as described above or described as preferred, at least one compound of the formula A and/or B, as described above or preferably described, or a mixture according to the invention, as described above or described as preferred.

The preparation, as described above or preferably described, preferably comprises a compound of the formula (I) and an antioxidant in a weight ratio of 1:1 to 1:20, preferably in a weight ratio of 1:4 to 1:15, particularly preferably in a weight ratio of 1:9. If a plurality of compounds of the formula (I) or a plurality of antioxidants are employed together, the total amount is determinant for the weight ratio. The preparation, as described above or preferably described, preferably comprises the mixture according to the invention in an amount of 0.01 percent by weight to 20 percent by weight, preferably 0.5% by weight-10% by weight, particularly preferably 0.75% by weight to 2% by weight, very particularly preferably one percent by weight. All amounts given are based on the total amount of the preparation.

The preparation according to the invention preferably comprises at least one organic or inorganic UV filter.

The preparations here are preferably cosmetic or dermatological preparations or medicinal products which can be applied topically. In this case, the preparations comprise a cosmetically or dermatologically suitable vehicle and, depending on the desired property profile, optionally further suitable ingredients. In the case of a medicinal product, a vehicle which is tolerated for the medicinal product is selected.

Can be applied topically in the sense of the invention means that the preparation is applied externally and locally, i.e. that the preparation must be suitable for, for example, application to the skin.

In the sense of the present invention, the term composition or formulation is also used synonymously alongside the term preparation.

The preparations may include or comprise, essentially consist of or consist of the said requisite or optional constituents. All compounds or components which can be used in the preparations are either known and commercially available or can be synthesised by known processes.

Further preferred combinations of embodiments are disclosed in the claims.

Preference is given in accordance with the invention to cosmetic or dermatological preparations. Particular preference is given in accordance with the invention to cosmetic preparations.

The invention furthermore relates to the preparation, as described above, comprising at least one organic or inorganic UV filter.

The invention furthermore relates to the preparation, as described above, characterised in that it comprises a cosmetic or dermatological vehicle or a vehicle which is tolerated for medicinal products.

The invention furthermore relates to a process for the preparation of the preparation, as described above, characterised in that the at least one compound of the formula (I), as described above or preferably described, at least one antioxidant or a mixture, as described above or preferably described, and a vehicle, is mixed, optionally with an organic and/or inorganic UV filter and optionally with further active substances or assistants.

The preparations may preferably comprise assistants, such as, for example, cosmetic oils (for example Caprylic/Capric Triglycerides, C12-15 Alkyl Benzoate, isopropyl myristate, Arylalkyl Benzoate, such as, for example, phenethyl benzoate (X-Tend 226) or oil components of the Cosmacol brand, such as Dimyristyl Tartrate, Tri C14-C15 Alkyl Citrate, C12-C13 Alkyl Lactate, Tridecyl Salicylate, C12-C13 Alkyl Octanoate, C12-C13 Alkyl Malate, C12-C13 Alkyl Citrate, C12-C13 Alkyl Tartrate), or polar-protic assistants (for example propylene glycol, glycerin, isopropanol, ethanol) or so-called solubilisers (for example butylphthalimides, isopropylphthalimides, dimethylisosorbides). Very particularly preferred cosmetic oils are C12-C13 Alkyl Lactate, commercially available as Cosmacol ELI, and phenethyl benzoate, commercially available as X-Tend 226.

The preparations described may furthermore also comprise coloured pigments, where the layer structure of the pigments is not limited. The coloured pigment should preferably be skin-coloured or brownish on use of 0.5 to 5% by weight. The choice of a corresponding pigment is familiar to the person skilled in the art.

The preparations described may, as described above, comprise hydrophilic or lipophilic sun-protection filters, which are effective in the UVA region and/or UVB region and/or IR and/or VIS region (absorbers). These substances can be selected, in particular, from cinnamic acid derivatives, salicylic acid derivatives, camphor derivatives, triazine derivatives, β,β-diphenylacrylate derivatives, p-aminobenzoic acid derivatives and polymeric filters and silicone filters, which are described in the application WO-93/04665. Further examples of organic filters are indicated in the patent application EP-A 0 487 404 and WO2009/077356.

Suitable organic UV-protecting substances can preferably be selected from the following list:

para-aminobenzoic acid and derivatives thereof: PABA, ethyl PABA, ethyldihydroxypropyl PABA, ethylhexyldimethyl PABA, for example marketed by ISP under the name “Escalol 507”, glyceryl PABA, PEG-25 PABA, for example marketed by BASF under the name “Uvinul P25”.

Salicylates: Homosalate, marketed by Merck under the name “Eusolex HMS”; ethylhexyl salicylate, for example marketed by Symrise under the name “Neo Heliopan OS”, dipropylene glycol salicylate, for example marketed by Scher under the name “Dipsal”, TEA salicylate, for example marketed by Symrise under the name “Neo Heliopan TS”.

β,β-Diphenylacrylate derivatives: Octocrylene, for example marketed by Merck under the name “Eusolex® OCR”, “Uvinul N539” from BASF, Etocrylene, for example marketed by BASF under the name “Uvinul N35”. Furthermore, for example, methoxycrylene, marketed by Hallstar under the name Solastay S1.

Benzophenone derivatives: Benzophenone-1, for example marketed under the name “Uvinul 400”; Benzophenone-2, for example marketed under the name “Uvinul D50”; Benzophenone-3 or Oxybenzone, for example marketed under the name “Uvinul M40”; Benzophenone-4, for example marketed under the name “Uvinul MS40”; Benzophenone-9, for example marketed by BASF under the name “Uvinul DS-49”, Benzophenone-5, Benzophenone-6, for example marketed by Norquay under the name “Helisorb 11”, Benzophenone-8, for example marketed by American Cyanamid under the name “Spectra-Sorb UV-24”, Benzophenone-12 n-hexyl 2-(4-diethylamino-2-hydroxybenzoyl) benzoate or 2-hydroxy-4-methoxybenzophenone, marketed by Merck, Darmstadt, under the name Eusolex® 4360.

Benzylidenecamphor derivatives: 3-Benzylidenecamphor, for example marketed under the name “Mexoryl SD” by Chimex, 4-Methylbenzylidenecamphor, for example marketed by Merck under the name “Eusolex 6300”, benzylidenecamphorsulfonic acid, for example marketed by Chimex under the name “Mexoryl SL”, Camphorbenzalkonium methosulfate, for example marketed by Chimex under the name “Mexoryl SO”, terephthalylidenedicamphorsulfonic acid, for example marketed by Chimex under the name “Mexoryl SX”, Polyacrylamidomethylbenzylidenecamphor marketed by Chimex under the name “Mexoryl SW”.

Phenylbenzimidazole derivatives: phenylbenzimidazolesulfonic acid, for example marketed by Merck under the name “Eusolex 232”, disodium phenyldibenzimidazoletetrasulfonate, for example marketed by Symrise under the name “Neo Heliopan AP”.

Phenylbenzotriazole derivatives: Drometrizoletrisiloxane, for example marketed by Rhodia Chimie under the name “Silatrizole”, Methylenebis(benzotriazolyl)tetramethylbutylphenol in solid form, for example marketed by Fairmount Chemical under the name “MIXXIM BB/100”, or in micronised form as an aqueous dispersion, for example marketed by BASF under the name “Tinosorb M”.

Triazine derivatives: Ethylhexyltriazone, for example marketed by BASF under the name “Uvinul T150”, Diethylhexylbutamidotriazone, for example marketed by Sigma 3V under the name “Uvasorb HEB”.

Further triazine derivatives are by way of example 2,4,6-tris(diisobutyl 4′-aminobenzalmalonate)-s-triazine or 2,4,6-tris(biphenyl)-1,3,5-triazine, or butyl 4-({4-{[4-(butoxycarbonyl)phenyl]amino}-6-[(3-{1,3,3,3-tetramethyl-1-[(trimethylsilyl)oxy]disiloxanyl}propyl)amino]-1,3,5-triazin-2-yl}amino)benzoate, marketed under the name Mexoryl SBS. Structure of Mexoryl SBS:

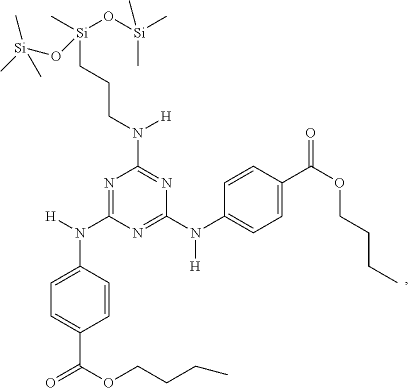 [SMILES: CCCCOC(=O)c1ccc(Nc2nc(NCCC[Si](C)(O[Si](C)(C)C)O[Si](C)(C)C)nc(Nc3ccc(C(=O)OCCCC)cc3)n2)cc1]

and Bis-ethylhexyloxyphenolmethoxyphenyltriazine, for example marketed by BASF under the name Tinosorb S.

Anthraniline derivatives: Menthyl anthranilate, for example marketed by Symrise under the name “Neo Heliopan MA”.

Imidazole derivatives: ethylhexyldimethoxybenzylidenedioxoimidazoline propionate.

Benzalmalonate derivatives: polyorganosiloxanes containing functional benzalmalonate groups, such as, for example, polysilicone-15, for example marketed by Hoffmann LaRoche under the name “Parsol SLX”.

4,4-Diarylbutadiene derivatives: 1,1-Dicarboxy(2,2′-dimethylpropyl)-4,4-diphenylbutadiene.

Benzoxazole derivatives: 2,4-bis[5-(1-dimethylpropyl)benzoxazol-2-yl(4-phenyl)imino]-6-(2-ethylhexyl)imino-1,3,5-triazine, for example marketed by Sigma 3V under the name Uvasorb K2A, and mixtures comprising this.

Piperazine derivatives, such as, for example, the compound

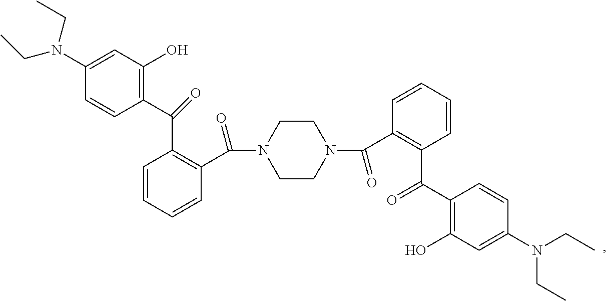 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

or the UV filters of the following structures

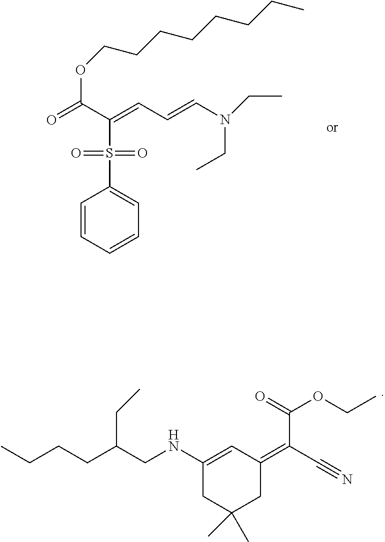

Preference is also given to a combination with UV filters based on polysiloxane copolymers having a random distribution in accordance with the following formula, where, for example, a=1.2; b=58 and c=2.8:

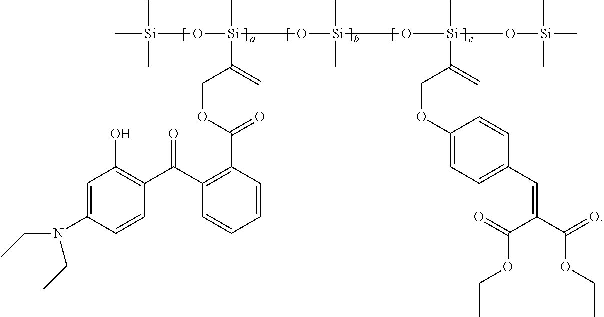 [SMILES: C=C(COC(=O)c1ccccc1C(=O)c1ccc(N(CC)CC)cc1O)[Si](C)(O[Si](C)(C)C)O[Si](C)(C)O[Si](C)(O[Si](C)(C)C)C(=C)COc1ccc(C=C(C(=O)OCC)C(=O)OCC)cc1]

These organic UV filters are generally incorporated into formulations in an amount of 0.01 percent by weight to 20 percent by weight, preferably 1% by weight-10% by weight.

The preparations may furthermore comprise inorganic UV filters, so-called particulate UV filters.

Conventional methods for the aftertreatment (or synonymously for the coating) of particulate UV filters are described, for example, in Cosmetics & Toiletries, February 1990, Vol. 105, pp. 53-64. One or more of the following aftertreatment components can be selected here: amino acids, beeswax, fatty acids, fatty acid alcohols, anionic surfactants, lecithin, phospholipids, sodium, potassium, zinc, iron or aluminium salts of fatty acids, polyethylenes, silicones, proteins (particularly collagen or elastin), alkanolamines, silicon dioxide, aluminium oxide, further metal oxides, phosphates, such as sodium hexametaphosphate, or glycerin.

Particulate UV filters which are preferably to be employed here are:
untreated titanium dioxides, such as, for example, the products Microtitanium Dioxide MT 500 B from Tayca; titanium dioxide P25 from Degussa,
Aftertreated titanium dioxides with aluminium oxide and silicon dioxide aftertreatment, such as, for example, the product “Microtitanium Dioxide MT 100 SA” from Tayca; or the product “Tioveil Fin” from Uniqema,
Aftertreated titanium dioxides with aluminium oxide and/or aluminium stearate/laurate aftertreatment, such as, for example, Microtitanium Dioxide MT 100 T from Tayca,
Aftertreated titanium dioxides with iron oxide and/or iron stearate aftertreatment, such as, for example, the product “Microtitanium Dioxide MT 100 F” from Tayca,
Aftertreated titanium dioxides with silicon dioxide, aluminium oxide and silicone aftertreatment, such as, for example, the product “Microtitanium Dioxide MT 100 SAS” from Tayca,
Aftertreated titanium dioxides with sodium hexametaphosphates, such as, for example, the product “Microtitanium Dioxide MT 150 W” from Tayca,
aftertreated titanium dioxides with aluminium oxide and finally with a manganese-containing layer, such as, for example, the product Eusolex T-PRO from Merck,
aftertreated titanium dioxides with silicon dioxide, such as, for example, the product Eusolex T-AVO,
aftertreated titanium dioxides with aluminium oxide and simeticones, such as, for example, the product Eusolex T-2000 from Merck,
aftertreated titanium dioxides with aluminium oxide and stearic acid, such as, for example, the product Eusolex T-S from Merck or
aftertreated titanium dioxides with aluminium oxide or silicon dioxide and monocetyl phosphate, such as, for example, the product Eusolex T-EASY from Merck.

The treated titanium dioxides to be employed for the combination may also be aftertreated with:
octyltrimethoxysilanes; such as, for example, the product Tego Sun T 805 from Degussa,
silicon dioxide; such as, for example, the product Parsol T-X from DSM,
aluminium oxide and stearic acid; such as, for example, the product UV-Titan M160 from Sachtleben,
aluminium and glycerin; such as, for example, the product UV-Titan from Sachtleben,
aluminium and silicone oils, such as, for example, the product UV-Titan M262 from Sachtleben,
sodium hexametaphosphate and polyvinylpyrrolidone, polydimethylsiloxanes, such as, for example, the product 70250 Cardre UF TiO2SI3 from Cardre,
polydimethylhydrogenosiloxanes, such as, for example, the product Microtitanium Dioxide USP Grade Hydrophobic from Color Techniques.

The treated zinc oxides to be employed for the combination can be selected, for example, from the following group:
Untreated zinc oxides, such as, for example, the product Z-Cote from BASF (Sunsmart), Nanox from Elementis
Aftertreated zinc oxides, such as, for example, the following products:
   ZnO aftertreated with polymethylhydrogenosiloxanes,
   Nanogard Zinc Oxide FN from Nanophase Technologies
   “SPD-Z1” from Shin-Etsu (ZnO aftertreated with a silicone-grafted acrylic polymer, dispersed in cyclodimethylsiloxanes)
   “Escalol Z100” from ISP (aluminium oxide-aftertreated ZnO dispersed in an ethylhexyl methoxycinnamate/PVP-hexadecene/methicone copolymer mixture)
   “Fuji ZNO-SMS-10” from Fuji Pigment (ZnO aftertreated with silicon dioxide and polymethylsilsesquioxane)
   Untreated cerium oxide micropigment, for example with the name “Colloidal Cerium Oxide” from Rhone Poulenc
   Untreated and/or aftertreated iron oxides with the name Nanogar from Arnaud.

By way of example, it is also possible to employ mixtures of various metal oxides, such as, for example, titanium dioxide and cerium oxide, with and without aftertreatment, such as, for example, the product Sunveil A from Ikeda.

These combinations with particulate UV filters are possible both as powder and also as dispersion or paste.

Preference is given here both to those from the group of the titanium dioxides, such as, for example, coated titanium dioxide (for example Eusolex® T-2000, Eusolex® T-AQUA, Eusolex® T-AVO, Eusolex® T-EASY), zinc oxides (for example Sachtotec®), iron oxides or also cerium oxides and/or zirconium oxides.

These inorganic UV filters are generally incorporated into the preparations in an amount of 0.1 percent by weight to 25 percent by weight, preferably 2% by weight-10% by weight.

By combination of one or more of the said compounds having a UV filter action, the protective action against harmful effects of the UV radiation can be optimised.

All said UV filters can also be employed in encapsulated form. In particular, it is advantageous to employ organic UV filters in encapsulated form.

Preferred preparations may also comprise at least one further cosmetic active compound, for example selected from self-tanners, anti-ageing active compounds, anti-wrinkle, anti-flake, anti-acne active compounds, deodorising active compounds, anti-cellulite active compounds, skin-lightening active compounds or vitamins.

Suitable self-tanners are dihydroxyacetome. glycerolaldehyde, hydroxymethylglyoxal, γ-dialdehyde, erythrulose, 6-aldo-D-fructose, ninhydrin, 5-hydroxy-1,4-naphtoquinone (juglone) or 2-hydroxy-1,4-naphtoquinone (lawsone) or a mixture of the said compounds. Self-tanners of this type are based on the action principle of the Maillard reaction.

Furthermore, the preparations may comprise as further ingredient at least one self-tanner which stimulates melanogenesis of the skin cells. An advantageous self-tanner from this class is, for example, 5-hydroxy-2-methyl-4-oxo-4H-chromen-7-yl hexadecanoate, which is marketed by Merck under the trade name RonaCare® Bronzyl.

Suitable anti-aging active compounds, in particular for skin-care preparations, are preferably so-called compatible solutes.

The compatible solutes employed are preferably substances selected from the group consisting of pyrimidinecarboxylic acids (such as ectoin and hydroxyectoin), proline, betaine, glutamine, cyclic diphosphoglycerate, N.-acetylornithine, trimethylamine N-oxide dimyo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) or/and dimannosyl diinositol phosphate (DMIP) or an optical isomer, derivative, for example an acid, a salt or ester, of these compounds, or combinations thereof.

Anti-ageing active compounds which can be used are additionally products from Merck, such as, for example, 5,7-dihydroxy-2-methyl-chromone, marketed under the trade name RonaCare®Luremine, or RonaCare®Cyclopeptide 5.

Suitable skin-lightening active compounds are melanogenesis inhibitors, such as, for example, hydroquinone, niacinamide, arbutin, alpha-arbutin, beta-arbutin, deoxyarbutin, vitamin C, ascorbyl palmitate, magnesium ascorbyl phosphate, sodium ascorbyl phosphate, azelaic acid, potassium 4-methoxysalicylate, trans-4-(aminomethyl)-cyclohexanecarboxylic acid (tranexamic acid), liquorice extract, ascorbyl glucoside, 3-O-ethylascorbyl ether, 2-O-ethylascorbyl ether, kojic acid, kojic acid dipalmitate, resorcinol, glutathione, cysteine, mulberry tree extract, hydrogen peroxide, zinc peroxide, sodium peroxide, benzoyl peroxide, glucosamine, aloesin, oleic acid 4-isopropyl-catechol, emblica, glycolic acid, ellagic acid, ferulaic acid, resveratrol, oxyresveratrol, quercetin, kaempferol, bile acid, hesperidin, p-coumaric acid, epicatechol gallate, epigallocatechol gallate, α-hydroxycarboxylic acids (for example lactic acid, salicylic acid, malic acid), α-tocopherol, (E)-3-(4-methoxyphenyl)acrylic acid (R)-2-((R)-3,4-dihydroxy-5-oxo-2,5-dihydro-furan-2-yl)-2-hydroxyethyl ester or 1-[4-(1,1-dimethylethyl)phenyl]-3-(4-methoxyphenyl)-1,3-propanediol. Preferred melanogenesis inhibitors are ascorbic acid and derivatives thereof, niacinamide, emblica, ellagic acid, mulberry tree extract, kojic acid, liquorice extract, rucinol, hydroquinone, azelaic acid, arbutin, magnesium ascorbyl phosphate (E)-3-(4-methoxy-phenyl)acrylic acid (R)-2-((R)-3,4-dihydroxy-5-oxo-2,5-dihydrofuran-2-yl)-2-hydroxyethyl ester or 1-[4-(1,1-dimethylethyl)-phenyl]-3-(4-methoxyphenyl)-1,3-propanediol.

The preparations to be employed may comprise vitamins as further ingredients.

Preference is given to vitamins and vitamin derivatives selected from vitamin B, thiamine chloride hydrochloride (vitamin B₁), riboflavin (vitamin B₂), nicotinamide, vitamin D, ergocalciferol (vitamin D₂), vitamin K₁, esculin (vitamin P active compound), thiamine (vitamin B₁), nicotinic acid (niacin), pyridoxine, pyridoxal, pyridoxamine, (vitamin B₆), panthothenic acid, biotin, folic acid and cobalamine (vitamin B₁₂), particularly preferably nicotinic acid, pantothenic acid and biotin. In the case of cosmetic application, vitamins are usually added with the preparations in ranges from 0.01 to 5.0% by weight, based on the total weight.

Retinoids are at the same time also effective anti-cellulite active compounds. A likewise known anti-cellulite active compound is caffeine.

Furthermore, the compounds of the formula (I) and the antioxidants can be combined in the preparations according to the invention with all active compounds and assistants as are listed systematically in WO2009/098139. In particular, these substances belong to the use categories stated therein of “Moisturizers and Humectants”, “Desquamating agents”, “Agents for improving the barrier function”, “Depigmenting Agents”, “Antioxidants”, “Dermo-relaxing or dermo-decontracting agents”, “Anti-glycation agents”, “Agents for stimulating the synthesis of dermal and/or epidermal macromolecules and/or for preventing their degradation”, “Agents for stimulating fibroblast or keratinocyte proliferation and/or keratinocyte differentiation”, “Agents for promoting the maturation of the horny envelope”, “NO-synthase inhibitors”, “Peripheral benzodiazepine receptor (PBR) antagonists”, “Agents for increasing the activity of the sebaceous glands”, “Agents for stimulating the energy metabolism of cells”, “Tensioning agents”, “Fat-restructuring agents”, “Sliming agents”, “Agents for promoting the cutaneous microcirculation”, “Calmatives or anti-irritants”, “Sebo-regulating or anti-seborrhoic agents”, “Astringents”, “Cicatrizing agents”, “Anti-inflammatory agents”, “Antiacne agents”. The contents of WO 2009/098139 expressly also belong to the disclosure content of the present application.

The said constituents of the preparation can be incorporated in the usual manner, with the aid of techniques which are well known to the person skilled in the art.

Suitable preparations are those for external application, for example can be sprayed onto the skin as cream or milk (O/W, W/O, O/W/O, W/O/W), as lotion or emulsion, in the form of oily-alcoholic, oily-aqueous or aqueous-alcoholic gels or solutions. They can be in the form of solid sticks or formulated as an aerosol.

The following may preferably be mentioned as application form of the preparations to be employed: solutions, suspensions, emulsions, PIT emulsions, Pickering emulsions, pastes, ointments, gels, creams, lotions, powders, soaps, surfactant-containing cleansing preparations, oils, aerosols plasters, compresses, bandages and sprays.

Preferred assistants originate from the group of preservatives, stabilisers, solubilisers, colorants, odour improvers.

Ointments, pastes, creams and gels may comprise the customary vehicles which are suitable for topical application, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary readily volatile, liquefied propellants, for example chlorofluorocarbons, propane/butane or dimethyl ether. Compressed air can also advantageously be used.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerin fatty acid esters, polyethylene glycols and fatty acid esters of sorbitan, or mixtures of these substances.

Pickering emulsions are emulsifier-free emulsions with particulate constituents, for example zinc oxide or titanium dioxide, and the usual vehicles, such as solvents or solubilisers, for example water, ethanol, isopropanol, ethyl carbonate, ethyl acetate, benzyl alcohol, benzyl benzoate, propylene glycol, 1,3-butyl glycol, oils, in particular cottonseed oil, groundnut oil, corn oil, olive oil, castor oil and sesame oil.

A preferred solubiliser in general is 2-isopropyl-5-methylcyclohexane-carbonyl-D-alanine methyl ester.

Suspensions may comprise the customary vehicles, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerin, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerin fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic application forms are also lipsticks, lip-care sticks, powder make-up, emulsion make-up and wax make-up, and sunscreen, pre-sun and after-sun preparations.

The preferred preparation forms also include, in particular, emulsions.

Emulsions are advantageous and comprise, for example, the said fats, oils, waxes and other fatty substances, as well as water and an emulsifier, as usually used for a preparation of this type.

The lipid phase may advantageously be selected from the following group of substances:
mineral oils, mineral waxes
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerin, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or from the group of esters of aromatic carboxylic acid and saturated and/or unsaturated, branched and/or unbranched alcohols having a chain length of 3 to 30 C atoms.

The aqueous phase of the preparations to be employed optionally advantageously comprises alcohols, diols or polyols having a low carbon number, and ethers thereof, preferably ethanol, isopropanol, propylene glycol, glycerin, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerin, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

The preparation may comprise cosmetic adjuvants which are usually used in this type of preparation, such as, for example, thickeners, softeners, moisturisers, surface-active agents, emulsifiers, preservatives, antifoams, perfumes, waxes, lanolin, propellants, dyes and/or pigments, and other ingredients usually used in cosmetics.

Even without further comments, it is assumed that a person skilled in the art will be able to utilise the above description in the broadest scope. The preferred embodiments and examples should therefore merely be regarded as descriptive disclosure which is absolutely not limiting in any way. The complete disclosure content of all applications and publications mentioned above and below is incorporated into this application by way of reference. The percent by weight ratios of the individual ingredients in the preparations of the examples expressly belong to the disclosure content of the description and can therefore be utilised as features.

Further important features and advantages of the invention arise from the sub-claims and from the examples.

It goes without saying that the features mentioned above and still to be explained below can be used not only in the respective combination indicated, but also in other combinations or in isolation without leaving the framework of the present invention.

Preferred embodiments of the invention are described in the examples and are explained in greater detail in the following description without restricting the scope of the present invention.

### EXAMPLES

### Example 1

### Synthesis of the Compound of the Formula (III-1)

The chain-extended alcohol is prepared from the starting materials 2,2,3,3,3-pentafluoropropan-1-ol (ABCR) and butylene carbonate (TCI) in accordance with patent application DE 10 2009 030 846 A1. This intermediate is esterified using aconitic acid (Alfa Aesar) by the synthetic procedure described and then sulfonated in the final reaction step by means of an aqueous sodium hydrogensulfite solution (Merck KGaA).

### Example 2

### Synthesis of the Compound of the Formula (III-2)

The chain-extended alcohol is prepared from the starting materials 2,2,3,3,4,4,4-heptafluorobutan-1-ol (ABCR) and propylene carbonate (Merck KGaA) in accordance with patent application DE 10 2009 030 846. This intermediate is esterified using aconitic acid by the synthetic procedure described and then sulfonated in the final reaction step by means of an aqueous sodium hydrogensulfite solution.

### Example 3

### Determination of the (Photo)Antioxidative Action of Various Mixtures by Reduction of β-Carotene Degradation After Irradiation

Mixtures of the test substances, as described below, with β-carotene are applied to a support material and irradiated. The stabilising action on beta-carotene is determined by irradiating both the sample comprising test substance and also a comparative batch with beta-carotene without test substance. The photodegradation of beta-carotene is determined in comparison in each case with an analogous dark batch. A triple determination is carried out on PMMA plates (17.5×2.5×0.5 cm; Schonberg, roughness 2 μm). In each case, 2 μl/cm² are applied to the PMMA plates. The solvent used is o-xylene. The radiation dose in the Suntester Atlas CPS+ is 150 kJ/m² at a power of 500 W/m² (about 3 MED).

Equipment:

Analytical balance (Satorius Extend), ultrasonic bath (Bandelin), shaker (Heidolph Multi Reax), pipette (Transferpettor; Brand: Handystep Electronic), Suntester CPS+ (Atlas), spectrophotometer (Agilent Technologies, Cary 60).

Mixtures, Amounts Indicated in Percent by Weight

Mixture A: 0.5% solution of β-carotene in o-xylene

Mixture B: 0.5% solution of β-carotene in o-xylene containing 6 percent by weight of butylene glycol, based on the total amount of the mixture.

Mixture C: 0.5% solution of β-carotene in o-xylene with 6 percent by weight of butylene glycol and 0.3% of the compound of the formula (III-1), based on the total amount of the mixture.

Mixture D: 0.5% solution of β-carotene in o-xylene with 2.5 percent by weight of bis(2-ethylhexyl) 2-(4-hydroxy-3,5-dimethoxybenzyl)malonate (RonaCare® AP), based on the total amount of the mixture.

Mixture E: 0.5% solution of β-carotene in o-xylene with 2.5 percent by weight of bis(2-ethylhexyl) 2-(4-hydroxy-3,5-dimethoxybenzyl)malonate and 0.3 percent by weight of the compound of the formula (III-1), based on the total amount of the mixture.

Mixture F: 0.5% solution of β-carotene in o-xylene with 2.5 percent by weight of bis(2-ethylhexyl) 2-(4-hydroxy-3,5-dimethoxybenzyl)malonate and 0.3 percent by weight of 1-methoxy-2-propanol, based on the total amount of the mixture.

Mixture G: 0.5% solution of β-carotene in o-xylene with 2.5 percent by weight of bis(2-ethylhexyl) 2-(4-hydroxy-3,5-dimethoxybenzyl)malonate, 0.3 percent by weight of the compound of the formula (III-1) and 0.3% by weight of 1-methoxy-2-propanol, based on the total amount of the mixture.

Mixture H: 0.5% solution of β-carotene in o-xylene with 2.5 percent by weight of bis(2-ethylhexyl) 2-(4-hydroxy-3,5-dimethoxybenzylidene)malonate (Oxynex® ST) and 0.3 percent by weight of 1-methoxy-2-propanol, based on the total amount of the mixture.

Mixture I: 0.5% solution of β-carotene in o-xylene with 2.5 percent by weight of bis(2-ethylhexyl) 2-(4-hydroxy-3,5-dimethoxybenzylidene)malonate, 0.3 percent by weight of the compound of the formula (III-1) and 0.3 percent by weight of 1-methoxy-2-propanol, based on the total amount of the mixture.

Procedure:

Preparation of the measurement solutions:

Fresh preparation of A on each measurement day by dissolution of β-carotene in o-xylene by treatment with ultrasound for 10 minutes (Bandelin ultrasonic bath).

Mixtures B-I are prepared by weighing out the individual substances into the sample container and adding the stock solution A. In order to complete the homogenisation, the samples are shaken for 15 minutes with the aid of the Heidolph Multi Reax.

Preparation of the Measurement:

In each case, 2 μL/cm² of solution of each measurement solution A-I are applied uniformly to a total of 6 PMMA plates with the aid of a pipette.

3 plates are stored in the dark, 3 plates are irradiated with the aid of the Atlas Suntesters CPS+ (150 kJ/m² at a power of 500 W/m² corresponding to about 3 MED).

All plates are rinsed individually with 50 ml of isopropanol in a 50 ml measuring cylinder. The measuring cylinders are treated with ultrasound for 15 minutes.

Measurement of the Absorption Spectrum:

About 2 ml of each solution are introduced 3 times into a quartz cell, and the absorption is measured between 350 and 550 nm with the aid of an Agilent Cary 60. The absorption value at the wavelength of 452 nm (absorption maximum of β-carotene in isopropanol) is used for the calculation. It must be ensured that pure isopropanol is used the blank value of the measurement.

Calculation of the Degradation of β-Carotene in Stock Solution A (a):

A₀: absorption at 452 nm before irradiation:

Aᵢ: absorption at 452 nm after irradiation

a=(A₀−Aᵢ)/Aₒ

Calculation of the Degradation of β-Carotene in the Samples, Here for Example B (b):

B₀: absorption at 452 nm before irradiation:

Bᵢ: absorption at 452 nm after irradiation

b=(Bₒ−Bᵢ)/Bₒ

Calculation of the Protection of β-Carotene in the Samples Compared with Stock Solution A:

P=(a−b)/a

Table 1 summarises the results for the fluorosurfactant of the formula (III-1).

TABLE 1

| | | Degradation of | Protection | Influence of the |
|---|---|---|---|---|
| | Antioxidant | β-carotene in % | [%] | fluorosurfactant* |
| A | — | 70 | — | — |
| B | — | 66 | 7 | — |
| C | — | 67 | 5 | 2% |
| A | — | 76 | — | — |
| D | RonaCare | 27 | 65 | — |
| | AP | | | |
| E | RonaCare | 22 | 71 | 39% |
| | AP | | | |
| A | — | 70 | — | — |
| F | RonaCare | 35 | 50 | — |
| | AP | | | |
| G | RonaCare | 33 | 53 | 8% |
| | AP | | | |
| A | — | 70 | — | — |
| H | Oxynex ® | 39 | 45 | — |
| | ST | | | |
| I | Oxynex ® | 30 | 58 | 31% |
| | ST | | | |

*calculated for the sample without fluorosurfactant standardised to 1 (maximum degradation)

The result for mixture C confirms that the fluorosurfactant of the formula (III-1) has no influence on the stability of beta-carotene. The degradation of beta-carotene is minimally influenced by the assistant butylene glycol used.

The result for mixtures A, D and E compared with mixtures A, F and G shows that the assistant 1-methoxy-2-propanol has an influence on the stability of beta-carotene. Despite this, the use of the compound of the formula (III-1) leads to an increase in the efficacy of the antioxidant with an influence of 8% (mixture G). The compound of the formula (III-1) in a mixture with the antioxidant bis(2-ethylhexyl) 2-(4-hydroxy-3,5-dimethoxybenzyl)malonate (mixture E) leads to an increase in the efficacy of the antioxidant of 39%, based on the mixture without fluorosurfactant.

The result for mixtures A, H and I shows that, in spite of the destabilising influence of the assistant 1-methoxy-2-propanol, the efficacy of the compound bis(2-ethylhexyl) 2-(4-hydroxy-3,5-dimethoxybenzylidene)malonate is increased by 31%, based on the mixture without fluorosurfactant, by the presence of the compound of the formula (III-1).

Recipes for cosmetic preparations which comprise compounds according to Example 1 or 2 are shown by way of example below. Corresponding preparations can be prepared in the same way with all compounds according to the invention.

In addition, the INCI names of the commercially available compounds are indicated.

UV-Pearl, OMC stands for the preparation with the INCI name: Water (for EU: Aqua), Ethylhexyl Methoxycinnamate, Silica, PVP, Chlorphenesin, BHT; this preparation is commercially available from Merck KGaA, Darmstadt, under the name Eusolex® UV Pearl™ OMC. The other UV-Pearls indicated in the tables each have an analogous composition in which OMC has been replaced by the UV filters indicated.

### Formulation Example 1

### W/O Emulsions (Numbers in % by Weight)

TABLE 1

| | 1-1 | 1-2 | 1-3 | 1-4 |
|---|---|---|---|---|
| Titanium dioxide | | | | 5 |
| Di 2-ethylhexyl 4-hydroxy-3,5- | 1 | | | |
| dimethoxybenzylmalonate | | | | |
| Compound of the formula (III-1) | 0.1 | | | |
| Mixture of di 2-ethylhexyl 4-hydroxy-3,5- | | 1 | 2 | 2 |
| dimethoxybenzylmalonate and | | | | |
| compound (III-1) (9:1) | | | | |
| Zinc oxide | | | 5 | |
| UV-Pearl, OMC | 15 | 15 | 15 | 15 |
| Polyglyceryl-3-Dimerate | 3 | 3 | 3 | 3 |
| Cera Alba | 0.3 | 0.3 | 0.3 | 0.3 |
| Hydrogenated Castor Oil | 0.2 | 0.2 | 0.2 | 0.2 |
| Paraffinium Liquidum | 7 | 7 | 7 | 7 |
| Caprylic/Capric Triglyceride | 7 | 7 | 7 | 7 |
| Hexyl Laurate | 4 | 4 | 4 | 4 |
| PVP/Eicosene Copolymer | 2 | 2 | 2 | 2 |
| Propylene Glycol | 4 | 4 | 4 | 4 |
| Magnesium Sulfate | 0.6 | 0.6 | 0.6 | 0.6 |
| Tocopherol | 0.5 | | | |
| Tocopheryl Acetate | 0.5 | | | |
| Cyclomethicone | 0.5 | 0.5 | 0.5 | 0.5 |
| Propylparabene | 0.05 | 0.05 | 0.05 | 0.05 |
| Methylparabene | 0.15 | 0.15 | 0.15 | 0.15 |
| Water | to 100 | to 100 | to 100 | to 100 |
| | 1-5 | 1-6 | 1-7 | 1-8 |
| Titanium dioxide | 3 | | 2 | 5 |
| Benzylidene malonate polysiloxane | | 1 | 0.5 | |
| 2-Ethylhexyl 4-hydroxyphenylpropionate | 1 | 1 | 0.5 | |
| Di 2-ethylhexyl 4-hydroxy-3,5-dimethoxy- | | | 1 | 1 |
| benzylidenemalonate | | | | |
| Mixture of di 2-ethylhexyl 4-hydroxy-3,5- | 5 | 3 | 1 | 1 |
| dimethoxybenzylmalonate and the compound | | | | |
| of the formula (III-1) (9:1) | | | | |
| Polyglyceryl-3-Dimerate | 3 | 3 | 3 | |
| Cera Alba | 0.3 | 0.3 | 0.3 | 2 |
| Hydrogenated Castor Oil | 0.2 | 0.2 | 0.2 | |
| Paraffinium Liquidum | 7 | 7 | 7 | |
| Caprylic/Capric Triglyceride | 7 | 7 | 7 | |
| Hexyl Laurate | 4 | 4 | 4 | |
| PVP/Eicosene Copolymer | 2 | 2 | 2 | |
| Propylene Glycol | 4 | 4 | 4 | |
| Magnesium Sulfate | 0.6 | 0.6 | 0.6 | |
| Tocopherol | | | 0.5 | |
| Tocopheryl Acetate | | | | 1 |
| Cyclomethicone | 0.5 | 0.5 | 0.5 | |
| Propylparabene | 0.05 | 0.05 | 0.05 | 0.05 |
| Methylparabene | 0.15 | 0.15 | 0.15 | 0.15 |
| Dicocoyl Pentyerythrityl Citrate (and) Sorbitan | | | | 6 |
| Sesquioleate (and) Cera Alba (and) | | | | |
| Aluminium Stearate | | | | |
| PEG-7 Hydrogenated Castor Oil | | | | 1 |
| Zinc Stearate | | | | 2 |
| Oleyl Erucate | | | | 6 |
| Decyl Oleate | | | | 6 |
| Dimethicone | | | | 5 |
| Tromethamine | | | | 1 |
| Glycerin | | | | 5 |
| Allantoin | | | | 0.2 |
| water | to 100 | to 100 | to 100 | to 100 |
| | 1-9 | 1-10 | 1-11 | 1-12 |
| Titanium dioxide | | | 3 | 3 |
| Benzylidene malonate polysiloxane | | 1 | 1 | |
| Methylene Bis-Benztriazolyl | 1 | | | 1 |
| Tetramethylbutylphenol | | | | |
| Zinc oxide | 5 | 2 | | |
| 2-Ethylhexyl 4-hydroxyphenyl- | 3 | 3 | 3 | 6 |
| propionate | | | | |
| Mixture of di 2-ethylhexyl 4-hydroxy- | 2 | 2 | 2 | 2 |
| 3,5-dimethoxybenzylmalonate and the | | | | |
| compound of the formula (III-2) (9:1) | | | | |
| UV-Pearl, OCR | | | | 5 |
| Di 2-ethylhexyl 4-hydroxy-3,5- | 10 | | 2 | 5 |
| dimethoxybenzylidenemalonate | | | | |
| UV-Pearl, Homosalate, BP-3 | | 10 | | |
| UV-Pearl, Ethylhexyl salicylate, BP-3 | | | 10 | |
| BMDBM | | | | 2 |
| Polyglyceryl-3-Dimerate | 3 | 3 | 3 | 3 |
| Cera Alba | 0.3 | 0.3 | 0.3 | 0.3 |
| Hydrogenated Castor Oil | 0.2 | 0.2 | 0.2 | 0.2 |
| Paraffinium Liquidum | 7 | 7 | 7 | 7 |
| Caprylic/Capric Triglyceride | 7 | 7 | 7 | 7 |
| Hexyl Laurate | 4 | 4 | 4 | 4 |
| PVP/Eicosene Copolymer | 2 | 2 | 2 | 2 |
| Propylene Glycol | 4 | 4 | 4 | 4 |
| Magnesium Sulfate | 0.6 | 0.6 | 0.6 | 0.6 |
| Tocopherol | 0.5 | | | |
| Tocopheryl Acetate | | | 0.5 | |
| Phenethyl 3,4-dihydroxyphenyl- | 0.5 | 0.5 | 0.5 | 0.5 |
| propionate | | | | |
| Propylparabene | 0.05 | 0.05 | 0.05 | 0.05 |
| Methylparabene | 0.15 | 0.15 | 0.15 | 0.15 |
| Water | to 100 | to 100 | to 100 | to 100 |

### Formulation Example 2

### O/W Emulsions, Numbers in % by Weight

TABLE 2

| | 2-1 | 2-2 | 2-3 | 2-4 |
|---|---|---|---|---|
| Titanium dioxide | 5 | | 3 | 1 |
| Methylene Bis-Benztriazolyl | | 2 | | |
| Tetramethylbutylphenol | | | | |
| Phenethyl 3,4-dihydroxyphenylpropionate | | | 1 | |
| 2-Ethylhexyl 4-hydroxyphenylpropionate | | | | |
| Mixture of di 2-ethylhexyl 4-hydroxy-3,5- | 1 | 1 | 2 | 2 |
| dimethoxybenzylmalonate and the | | | | |
| compound of the formula (III-1) (9:1) | | | | |
| Di 2-ethylhexyl 4-hydroxy-3,5-dimethoxy- | | | 1 | |
| benzylidenemalonate | | | | |
| Di 2-ethylhexyl 2-cyano-3,3,-diphenyl- | 4 | 7 | 1 | 2 |
| propionate | | | | |
| 4-Methylbenzylidene Camphor | 3 | 3 | | |
| BMDBM | | 3 | | |
| Phenylbenzimidazole Sulfonic Acid | | | | 4 |
| Stearyl Alcohol (and) Steareth-7 (and) | 3 | 3 | 3 | |
| Steareth-10 | | | | |
| Glyceryl Stearate (and) Ceteth-20 | 3 | 3 | 3 | |
| Glyceryl Stearate | 3 | 3 | 3 | |
| Microwax | 1 | 1 | 1 | |
| Cetearyl Octanoate | 11.5 | 11.5 | 11.5 | |
| Caprylic/Capric Triglyceride | 6 | 6 | 6 | 14 |
| Oleyl Oleate | 6 | 6 | 6 | |
| Propylene Glycol | 4 | 4 | 4 | |
| Glyceryl Stearate SE | | | | 6 |
| Stearic Acid | | | | 2 |
| Persea Gratissima | | | | 8 |
| Propylparabene | 0.05 | 0.05 | 0.05 | 0.05 |
| Methylparabene | 0.15 | 0.15 | 0.15 | 0.15 |
| Tromethamine | 1.8 | | | 1.8 |
| Glycerin | | | | 3 |
| Water | to 100 | to 100 | to 100 | to 100 |

### Formulation Example 3

### Gels, Numbers in % by Weight

TABLE 3

| | 3-1 | 3-2 | 3-3 | 3-4 | 3-5 | 3-6 |
|---|---|---|---|---|---|---|
| Titanium dioxide | | 2 | | | | |
| 5,6,7-Trihydroxyflavone | | | 1 | | | 1 |
| Di 2-ethylhexyl 4-hydroxy-3,5- | | | 1 | | | |
| dimethoxybenzylidenemalonate | | | | | | |
| Mixture of di 2-ethylhexyl 4-hydroxy- | 1 | 1 | 1 | 2 | 2 | 1 |
| 3,5-dimethoxybenzylmalonate and the | | | | | | |
| compound of the formula (III-2) (9:1) | | | | | | |
| Di 2-ethylhexyl 2-cyano-3,3,- | 5 | 5 | 5 | 5 | 5 | 5 |
| diphenylpropionate | | | | | | |
| 2-Ethylhexyl 4-hydroxyphenyl- | 1 | 5 | | | | 2 |
| propionate | | | | | | |
| Benzylidene malonate polysiloxane | | | 1 | | | 1 |
| Methylene Bis-Benztriazolyl | | 1 | | 1 | 1 | |
| Tetramethylbutylphenol | | | | | | |
| Zinc oxide | | | 2 | | 5 | 2 |
| UV-Pearl, Ethylhexyl Mehtoxycinnamat | 30 | 15 | 15 | 15 | 15 | 15 |
| Butylmethoxydibenzoylmethane | | 1 | | | | |
| Prunus Dulcis | 5 | 5 | 5 | 5 | 5 | 5 |
| Tocopheryl Acetate | 0.5 | | | | | |
| Caprylic/Capric Triglyceride | 3 | 3 | 3 | 3 | 3 | 3 |
| Octyldodecanol | 2 | 2 | 2 | 2 | 2 | 2 |
| Decyl Oleate | 2 | 2 | 2 | 2 | 2 | 2 |
| PEG-8 (and) Tocopherol (and) Ascorbyl | 0.05 | 0.05 | 0.05 | 0.05 | 0.05 | 0.05 |
| Palmitate (and) Ascorbic Acid (and) | | | | | | |
| Citric Acid | | | | | | |
| Sorbitol | 4 | 4 | 4 | 4 | 4 | 4 |
| Polyacrylamide (and) C13-14 | 3 | 3 | 3 | 3 | 3 | 3 |
| Isoparaffin (and) Laureth-7 | | | | | | |
| Propylparabene | 0.05 | 0.05 | 0.05 | 0.05 | 0.05 | 0.05 |
| Methylparabene | 0.15 | 0.15 | 0.15 | 0.15 | 0.15 | 0.15 |
| Water | to 100 | to 100 | to 100 | to 100 | to 100 | to 100 |

### Formulation Example 4

### Hair Mascara

| Ingredients | [%] |
|---|---|
| A | |
| PEARLESCENT PIGMENT | 20.0 |
| B | |
| CETEARETH-25 | 1.8 |
| CETEARYL ALCOHOL | 5.0 |
| DIMETHICONE | 1.0 |
| MIXTURE OF DI 2-ETHYLHEXYL 4- | 1.0-2.0 |
| HYDROXY-3,5-DIMETHOXY BENZYL | |
| MALONATE AND THE COMPOUND OF | |
| THE FORMULA (III-1) (9:1) | |
| C | |
| AQUA (WATER) | to 100 |
| POLYQUATERNIUM-16 | 0-2.5 |
| PROPYLENE GLYCOL | 1.8 |
| D | |
| AQUA (WATER) | 9.5 |
| HYDROXYPROPYLCELLULOSE | 0.5 |
| E | |
| AQUA (WATER) | 9.5 |
| MAGNESIUM ALUMINIUM SILICATE | 0.5 |
| IMIDAZOLIDINYL UREA | 0.3 |

Preparation Process:

Heat phase B to 75° C., phase C to 80° C. Slowly add phase B to phase C with stirring. Cool to 65° C. with stirring and homogenise. Cool to 40° C. and add phases D, E and F to phase B/C with stirring and homogenise again. Then add the pearlescent pigment with stirring. Cool to room temperature and adjust the pH to 6.0-6.5.

### Formulation Example 5

### Conditioner with IR3535®

| Ingredients | [%] |
|---|---|
| A | |
| ETHYLBUTYL ACETYLAMINOPROPIONATE | 10.0 |
| PVP/VA COPOLYMER | 4.0 |
| PERFUME | 0.3 |
| QUATERNIUM-80 | 0-2.5 |
| PEG-40 HYDROGENATED CASTOR OIL | 1.0 |
| ALCOHOL | 15.0 |
| MIXTURE OF DI 2-ETHYLHEXYL 4- | 1.0-3.0 |
| HYDROXY-3,5-DIMETHOXYBENZYL- | |
| MALONATE AND THE COMPOUND OF THE | |
| FORMULA (III-1) (9:1) | |
| B | |
| CETRIMONIUM CHLORIDE | 0.5 |
| AQUA (WATER) | To 100 |
| C | |
| COCAMIDOPROPYL BETAINE | 4.0 |

Preparation process: Mix phases A and B separately. Add phase B to phase A with stirring. Add phase C.

### Formulation Example 6

### Hair Conditioner with Pearlescent Pigment

| Ingredients | [%] |
|---|---|
| A | |
| PEARLESCENT PIGMENT | 3.00 |
| DISODIUM EDTA | 0.05 |
| AQUA (WATER) | to 100 |
| B | |
| CETEARYL ALCOHOL, BEHENTRIMONIUM | 5.00 |
| METHOSULFATE | |
| OCTYLDODECANOL | 1.10 |
| CETYL ALCOHOL | 1.00 |
| GLYCERIN | 1.00 |
| BEHENTRIMONIUM CHLORIDE | 0.70 |
| METHOXY PEG/PPG-7/3 AMINOPROPYL | 0.70 |
| DIMETHICONE | |
| QUATERNIUM-80 | 0-2.5 |
| MIXTURE OF DI 2-ETHYLHEXYL 4-HYDROXY- | 1.0-3.0 |
| 3,5-DIMETHOXYBENZYLIDENEMALONATE | |
| AND THE COMPOUND OF THE FORMULA | |
| (III-1) (9:1) | |
| C | |
| COCODIMONIUM HYDROXYPROPYL- | 0.70 |
| SILICAMINO ACIDS | |
| PHENOXYETHANOL, BENZOIC ACID, | 0.40 |
| DEHYDROACETIC ACID | |
| CITRIC ACID | 0.20 |
| PERFUME | 0.60 |

Preparation Process:

Disperse the pearlescent pigment and Titriplex III in the water of phase A. Heat the constituents of phases A and B to 75° C. Add phase B to phase A with stirring and homogenise. Cool to 40° C. and add the constituents of phase C. Cool to 30° C. and homogenise again for about 30 sec. Adjust the pH to 3.6-4.0.

Notes: recommended pearlescent pigments are TIMIRON® silver pigments and TIMIRON® interference pigments from Merck.

### Formulation Example 7

### O/W Tanning Cream

| INCI | [WT-%] |
|---|---|
| A | |
| GLYCERYL STEARATE, STEARETH-25- | 8 |
| CETETH-20, STEARYL ALCOHOL | |
| CAPRYLIC/CAPRIC TRIGLYCERIDE | 3 |
| ISOPROPYL MYRISTATE | 2 |
| PARAFFINUM LIQUIDUM (MINERAL OIL) | 12 |
| MIXTURE OF DI 2-ETHYLHEXYL 4-HYDROXY- | 2 |
| 3,5-DIMETHOXYBENZYLMALONATE AND | |
| THE COMPOUND OF THE FORMULA (III-1) | |
| (9:1) | |
| PARAFFIN | 2 |
| PROPYLPARABEN | 0.15 |
| DIHYDROXY METHYLCHROMONYL | 4 |
| PALMITATE | |
| B | |
| PROPYLENE GLYCOL | 4 |
| SORBITOL | 2 |
| METHYLPARABEN | 0.05 |
| AQUA (WATER) | TO 100 |
| C | |
| WATER, DEMINERALISED | 11.8 |
| D | |
| PERFUME | 0.50 |

Preparation Process:

Firstly, phases A and B are warmed separately to 75° C. Phase A is then slowly added to phase B with careful stirring. The mixture is homogenised at 65 C for one minute. The mixture is subsequently cooled to 40° C. with stirring, and phase C is added with stirring, cooled to 35° C. and phase B is added and cooled further.

Firstly, phases A and B are warmed to 80° C. Phase B is then slowly added to phase A with stirring and homogenised. The mixture is then cooled, and phase C is added at 40° C.

### Formulation Example 8

### O/W Tanning Lotion

| INCI | [WT-%] |
|---|---|
| A | |
| CETEARYL ALCOHOL, CETEARYL GLUCOSIDE | 4 |
| SORBITAN STEARATE | 1.5 |
| CETEARYL ALCOHOL | 1 |
| C12-13 ALIKYL LACTATE | 2 |
| ISOHEXADECANE | 1 |
| PARAFFINUM LIQUIDUM (MINERAL OIL) | 3 |
| MIXTURE OF DI 2-ETHYLHEXYL 4-HYDROXY- | 1-2 |
| 3,5-DIMETHOXYBENZYLIDENEMALONATE AND | |
| THE COMPOUND OF THE FORMULA (III-1) (9:1) | |
| CYCLOMETHICONE, DIMETHICONE | 2 |
| CROSSPOLYMER | |
| TOCOPHERYL ACETATE | 0.5 |
| PROPYLPARABEN | 0.05 |
| B | |
| ECTOIN | 0.5 |
| GLYCERIN | 2 |
| AQUA (WATER) | TO 100 |
| METHYLPARABEN | 0.15 |
| C | |
| XANTHAN GUM | 0.2 |
| D | |
| AQUA, ALCOHOL DENAT, LECITHIN, GLYCERIN, | 5 |
| DISODIUM PHOSPHATE | |
| DIHYDROXY METHYLCHROMONYL PALMITATE | 4 |
| AQUA (WATER) | 10 |
| E | |
| PERFUME | 0.2 |

Preparation Process:

Firstly, phases A and B are mixed separately and warmed to 75° C. Phase C is then added to phase B and added to phase A with stirring. The mixture is homogenised. The mixture is then cooled with stirring, and phases D and B are added at 40° C.

### Formulation Example 9

### O/W Tanning Cream with UV A/B Protection

| INCI | [WT-%] |
|---|---|
| A | |
| ETHYLHEXYL METHOXYCINNAMATE, BHT | 3 |
| BENZOPHENONE-3 | 0.5 |
| GLYCERYL STEARATE, STEARETH-25, | 8 |
| CETETH-20, STEARYL ALCOHOL | |
| MIXTURE OF DI 2-ETHYLHEXYL 4- | 1.0 |
| HYDROXY-3,5-DIMETHOXYBENZYL- | |
| MALONATE AND THE COMPOUND OF THE | |
| FORMULA (III-1) (9:1) | |
| CETEARYL ALCOHOL | 1.5 |
| CETEARYL OCTANOATE | 5 |
| CAPRYLIC/CAPRIC TRIGLYCERIDE | 5 |
| PARAFFINUM LIQUIDUM (MINERAL OIL) | 3 |
| STEAROXY DIMETHICONE | 1.6 |
| DIMETHICONE | 0.5 |
| PROPYLPARABEN | 0.05 |
| B | |
| PROPYLENE GLYCOL | 3 |
| SODIUM METHYLPARABEN | 0.17 |
| AQUA (WATER) | TO 100 |
| DIHYDROXY METHYLCHROMONYL | 5 |
| PALMITATE | |
| C | |
| DIHYDROXYACETONE | 5 |
| AQUA (WATER | 10 |

Preparation Process:

Firstly, phases A and B are mixed separately and warmed to 80° C. Phase B is then slowly added to phase A with stirring. The mixture is homogenised and cooled to 40° C., and phase C is added, then cooled to room temperature.

### Formulation Example 10

### Soft Cream Sunscreen (O/W)

| RAW MATERIAL (INCI) | WT.-% |
|---|---|
| A | |
| TITANIUM DIOXIDE | 10.0 |
| STEARETH-10, STEARETH-7, STEARYL | 2.0 |
| ALCOHOL | |
| GLYCERYL STEARATE, CETETH-20 | 2.0 |
| GLYCERYL STEARATE | 3.0 |
| MIXTURE OF DI 2-ETHYLHEXYL 2-(4- | 1.0 |
| METHOXY)-BENZYLIDENEMALONATE AND | |
| THE COMPOUND OF THE FORMULA (III-1) | |
| (9:1) | |
| MICROWAX | 1.0 |
| OLEYL OLEATE | 6.0 |
| CETEARYL OCTANOATE | 14.0 |
| CAPRYLIC/CAPRIC TRIGLYCERIDE | 4.0 |
| B | |
| PROPYLENE GLYCOL | 4.0 |
| ALLANTOIN | 0.2 |
| WATER | TO 100 |
| PRESERVATIVE | Q.S. |

Preparation:

Heat phase A and phase B to 80° C. Slowly add phase B to phase A with stirring, homogenise and cool with stirring.

### Formulation Example 11

### Sunscreen Lotion (O/W)

| RAW MATERIAL (INCI) | % |
|---|---|
| A | |
| OCTOCRYLENE | 6.0 |
| BUTYL METHOXYDIBENZOYLMETHANE | 2.0 |
| POLYGLYCERYL-3 METHYLGLUCOSE | 4.0 |
| DISTEARATE | |
| ETHYLHEXYL STEARATE | 8.0 |
| CETEARYL ISONONANOATE | 2.0 |
| PVP/EICOSENE COPOLYMER | 1.0 |
| MIXTURE OF 3-(3,4,5-TRIMETHOXY- | 1.0 |
| BENZYLLIDENE)-2,4-PENTANEDIONE AND | |
| THE COMPOUND OF THE FORMULA (III-1) | |
| (9:1) | |
| TOCOPHERYL ACETATE | 1.0 |
| B | |
| XANTHAN GUM | 0.3 |
| SODIUM CETEARYL SULFATE | 1.0 |
| GLYCERIN | 5.0 |
| WATER | TO 100 |
| C | |
| TITANIUM DIOXIDE | 4.0 |
| D | |
| PRESERVATIVE | Q.S. |

Preparation:

Heat phase A to 80° C. Pre-swell the Keltrol of phase B in the water, then add the remaining raw materials and heat to 80° C. Add phase A to phase B and homogenise for 2 min (hand mixer): cool with stirring and add phase C at 35° C. Homogenise again for 1 min (hand mixer). Cool to room temperature and stir in phase D.

### Formulation Example 12

### Sunscreen Spray with Tanning Enhancement

| RAW MATERIAL (INCI) | % |
|---|---|
| A) | |
| CERALUTION ® C; SASOL | 15.0 |
| B) | |
| TITANIUM DIOXIDE | 5.0 |
| ETHYLHEXYL METHOXYCINNAMATE | 4.8 |
| ETHYLHEXYL SALICYLATE | 4.8 |
| MIXTURE OF DI 2-ETHYLHEXYL 4-HYDROXY- | 1.0 |
| 3,5-DIMETHOXYBENZYLMALONATE AND THE | |
| COMPOUND OF THE FORMULA (III-1) (9:1) | |
| CYCLOMETHICONE | 1.0 |
| C12-15 ALKYL BENZOATE | 2.5 |
| TRIDECYL SALICYLATE | 2.5 |
| C) | |
| DIHYDROXYACETONE | 3 |
| WATER (AQUA), DEIONIZED | TO 100 |
| WATER (AQUA), DEIONIZED WITH 4% AVICEL | 25.0 |
| CL 611 (MICROCRYSTALLINE CELLULOSE (AND) | |
| CELLULOSE GUM) | |
| D) | |
| PRESERVATIVE | Q.S. |

Preparation: Slowly add phase B to phase A at room temperature with stirring. Then add phase C. Subsequently add phase D.

INCI CERALUTION® C:

Aqua (and) Capric/Caprylic triglyceride (and) Glycerin (and) Ceteareth-25 (and) Sodium Dicocoylethylenediamine PEG-15 Sulfate (and) Sodium Lauroyl Lactylate (and) Behenyl Alcohol (and) Glyceryl Stearate (and) Glyceryl Stearate Citrate (and) Gum Arabic (and) Xanthan Gum (and) Phenoxyethanol (and) Methylparaben (and) Ethylparaben (and) Butylparaben (and) Isobutylparaben

### Formulation Example 13

### Hair Toning Spray Pump

| | |
|---|---|
| A | |
| DYE | 1.0 |
| ETHANOL 96% EXTRA PURE | TO 100 |
| PVP/VA COPOLYMER, PVP/VA W 735 | 6.0 |
| B | |
| DIETHYLHEXYL SYRINGYLIDENEMALONATE, | 0.1 |
| CAPRYLIC/CAPRIC TRIGLYCERIDE | |
| (OXYNEX ® ST LIQUID) | |
| COMPOUND OF THE FORMULA (III-1) | 0.1 |
| PEG-75 LANOLIN BHT | 0.2 |
| (SOLAN E - LOW DIOXANE) | |
| PERFUME | 0.1 |
| (FRAG 280853 GREEN ACTIVATING) | |
| C | |
| WATER, DEMINERALISED | 13.0 |
| TITRIPLEX III | 0.1 |
| PEG-12 DIMETHICONE | 0.5 |
| DOW CORNING 193 FLUID | |
| 0.1% D&C RED NO 33 (CI 17200) IN WATER | 0.2 |
| PEG-40 HYDROGENATED CASTOR OIL | 1.0 |
| (CREMOPHOR RH 410) | |

Preparation: Pre-dissolve phase A until a clear solution is obtained. Add phase B to phase A with stirring. Pre-mix phase C and add to the remainder, stir until a homogeneous mixture has formed.

### Formulation Example 14

### Hair Care Formulation

| Component | Weight [%] |
|---|---|
| Disodium EDTA | 0.1 |
| Oxynex ® ST | 2.0 |
| Compound of the formula (III-1) | 0.1 |
| Dye | 1.5 |
| Cetyl pyridinium chloride | 0 |
| Pitera ® | 10 |
| Ascorbyl glycoside | 0 |
| Niacinamide | 5.0 |
| Polyquaternium 37 | 0 |
| Isohexadecane | 2.5 |
| Isopropyl isostearate | 1.3 |
| Sucrose polycottonseedate | 0.67 |
| Polymethylsilsesquioxane | 0.25 |
| Cetearyl glucoside + cetearyl alcohol | 0.2 |
| Behenyl alcohol | 0.4 |
| Ethylparaben | 0.2 |
| Propylparaben | 0.1 |
| Cetyl alcohol | 0.32 |
| Stearyl alcohol | 0.48 |
| Tocopheryl acetate | 0.5 |
| PEG-100 stearate | 0.1 |
| Glycerin | 7.0 |
| Titanium dioxide | 0.6 |
| Polyacrylamide + C13-14 isoparaffin + laureth-7 | 2.0 |
| Panthenol | 1.0 |
| Benzyl alcohol | 0.4 |
| Dimethicone + dimethiconol | 2.0 |
| Water | to 100 |

### Formulation Example 15

### Hair Rinse

| Component | Weight [%] |
|---|---|
| Cetearyl Alcohol | 10 |
| Sunflowerseedamidopropyl Ethyldimonium | 0.5 |
| Ethosulfate | |
| Ceteareth-20 | 3.0 |
| Panthenol | 0.4 |
| Phenyl Trimethicone | 0.3 |
| Hydroxypropyl Guar Hydroxypropyltrimonium | 0.8 |
| Chloride | |
| 4-Methoxycinnamic acid 6-O-ascorbate | 1.0 |
| Mixture of di 2-ethylhexyl 4-hydroxy-3,5- | 1.0 |
| dimethoxybenzylmalonate and the | |
| compound of the formula (III-1) (9:1) | |
| Passiflora Incarnata Seed Oil | 0.2 |
| Basic red 51 | 0.1 |
| Basic red 76 | 0.2 |
| Perfume | 1.0 |
| Preservative | q.s. |
| Citric Acid/Sodium Hydroxide | q.s. to pH 5.5 |
| Aqua | to 100 |

### Formulation Example 16

### Hair Rinse

| Component | Weight [%] |
|---|---|
| Cetearyl Alcohol | 5.0 |
| Cetrimonium Chloride | 1.0 |
| Polysilicone-15 | 0.5 |
| Panthenol | 0.4 |
| Dimethicone | 0.8 |
| Hydroxypropyl Guar Hydroxypropyltrimonium | 1.0 |
| Chloride | |
| 2,4,6-Trimethoxycinnamic acid 6-O-ascorbate | 0.5 |
| 4-Methoxycinnamic acid 6-O-ascorbate | 0.5 |
| Mixture of di 2-ethylhexyl 4-hydroxy-3,5- | 1.0 |
| dimethoxybenzylidenemalonate and the | |
| compound of the formula (III-2) (9:1) | |
| Hydrogenated Grapeseed Oil | 1.5 |
| Tiliroside | 0.05 |
| Avocado Extrakt | 0.5 |
| Perfume | 1.0 |
| Preservative | q.s. |
| Citric Acid/Sodium Hydroxide | q.s. to pH 5.5 |
| Aqua | to 100 |

### Formulation Example 17

### Hair Foam

| Component | Weight [%] |
|---|---|
| Quaternium-80 | 0.2 |
| Polyquaternium-11 | 0.7 |
| PEG-60 Hydrogenated Castor Oil | 0.5 |
| Perfume | 1.0 |
| Preservative | q.s. |
| 4-Methoxycinnamic acid 6-O-ascorbate | 0.3 |
| Mixture of di 2-ethylhexyl 4-hydroxy-3,5- | 1.0 |
| dimethoxybenzylmalonate and the | |
| compound of the formula (III-1) (9:1) | |
| Passiflora Incarnata Seed Oil | 0.2 |
| Citric Acid/Sodium Hydroxide | q.s. to pH 4.5 |
| Aqua | to 100 |

The formulation can be employed as a leave-on or rinse-off formulation and is packaged as an aerosol in the ratio formulation/propellant gas=90/10 in a pressure container. Propellant gases which can be employed are, for example, propane or butane or mixtures thereof.

### Formulation Example 18

### Shampoo

| Component | Weight [%] |
|---|---|
| Sodium Laureth Sulfate | 5.0 |
| Cocamidopropy Betaine | 5.0 |
| Lauroyl Glutamic Acid | 3.0 |
| Decyl Glucoside | 5.0 |
| Polyquaternium-10 | 0.5 |
| PEG-3 Distearate | 0.8 |
| 2,4,6-Trimethoxycinnamic acid 6-O-ascorbate | 0.5 |
| Mixture of di 2-ethylhexyl 4-hydroxy-3,5- | 1.0 |
| dimethoxybenzylmalonate and the | |
| compound of the formula (III-1) (9:1) | |
| Evening primrose oil | 0.3 |
| Basic Red 51 | 0.1 |
| Ubiquinone | 0.1 |
| Benzyl Alcohol | 0.5 |
| Perfume | 1.0 |
| Preservative | q.s. |
| Sodium Chloride | 0.8 |
| Citric Acid/Sodium Hydroxide | q.s. to pH 5.5 |
| Aqua | to 100 |

### Formulation Example 19

### Hair Dye Recipes

| Component | Weight [%] |
|---|---|
| Benzyl Alcohol | 2.5 |
| Propylene Carbonate | 10 |
| Ethanol | 5.0 |
| Hydroxyethylcellulose | 2.0 |
| Pirenoxin Sodium CAS 51410-30-1 | 2.0 |
| 4-Methoxycinnamic acid 6-O-ascorbate | 1.0 |
| Mixture of di 2-ethylhexyl 4-hydroxy-3,5- | 1.0 |
| dimethoxybenzylmalonate and the | |
| compound of the formula (III-1) (9:1) | |
| Perfume | 1.0 |
| Preservative | q.s. |
| Citric Acid | q.s. ad pH 5.5 |
| Aqua | to 100 |

1. A method for increasing the efficacy of an antioxidant, comprising bringing together said antioxidant with a compound of formula (I) 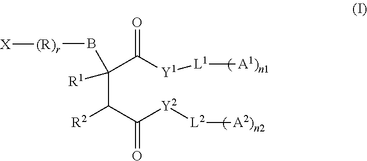
where
X is a hydrophilic group,
R is linear or branched alkylene having 1 to 12 C atoms, where one or more non-adjacent C atoms may be replaced by O, S and/or N,
r is 0 or 1,
B is a single bond, —O—, —NH—, —NR′—, —CH₂—, —C(O)—O—, —S—, —CH₂—O—, —O—C(O)—, —O—C(O)—O—, —N—C(O)—, —C(O)—N—, —O—C(O)—N—, —N—C(O)—N—, —Si(R′)₂—, —Si(R′)₂—O—, —O—SO₂— or —SO₂—O—,
R′ is a linear or branched alkyl group having 1 to 12 C atoms,
R¹ and R², independently of one another, are hydrogen or —CH₂—COY³-L³-(A³)ₙ₃,
Y¹,Y² and Y³, independently of one another, are O, S or N,
L¹, L² and L³, independently of one another, are linear or branched alkylene having 1 to 12 C atoms, where one or more non-adjacent C atoms may be replaced by O, S and/or N,
A¹, A² and A³, independently of one another, are hydrogen or a group of the structure —Zⁱ(CR³R⁴)ₘᵢRfⁱ, where i denotes 1, 2 or 3,
Zⁱ is O, S or N and is bonded to a C atom of Lⁱ or is a single bond,
R³ and R⁴, independently of one another, are hydrogen or a straight-chain or branched alkyl group having 1 to 12 C atoms,
Rfⁱ is a fluorine-containing radical,
n1, n2 and n3, independently of one another, are 1, 2 or 3,
m1, m2 and m3, independently of one another, are 0, 1, 2, 3, 4 or 5,
and the compounds of the formula (I) contain at least one Rfⁱ group.

2. The method according to claim 1, wherein the compound of formula (I) is a compound of formulae (II), (III) and/or (IV), 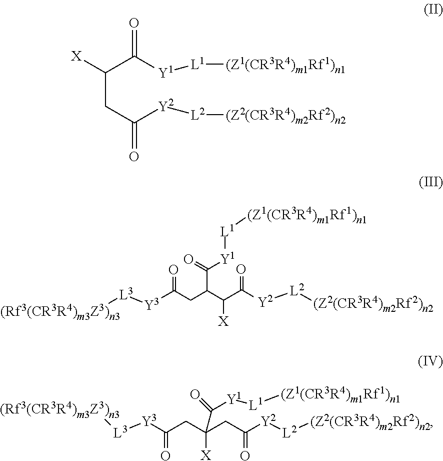
where
X stands for —SO₃⁻, —OSO₃⁻, —PO₃²⁻ or OPO₃²⁻,
Y¹, Y², Y³, Z¹, Z² and Z³ stand for O,
Rf¹, Rf² and Rf³ stand for perfluorinated groups Rfⁱ having 1 to 4 C atoms,
n1, n2 and n3 stand for 1 or 2,
m1, m2 and m3 stand for 0, 1, 2, 3 or 4,
L¹, L² and L³ stand for a linear or branched alkylene having 2 to 6 C atoms and
R³ and R⁴ stand for hydrogen or an alkyl group having 1 to 3 C atoms.

3. The method according to claim 1, wherein the compound of formula (I) is a compound of one of formulae (III-1) to (III-6):

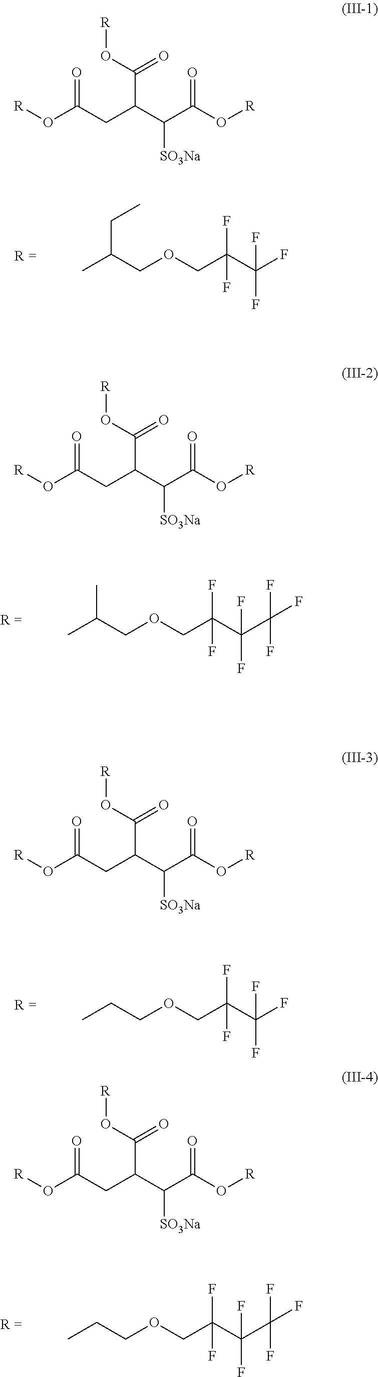

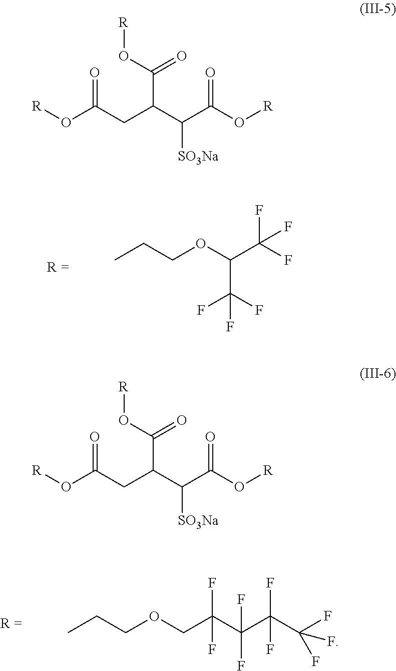

4. The method according to claim 1, wherein the efficacy of the antioxidants selected from the group amino acids, imidazoles, peptides, carotenoids, carotenes, chlorogenic acid and derivatives thereof, lipoic acid and derivatives thereof, aurothioglucose, thiols, thiodipropionic acid and derivatives thereof, sulfoximine compounds, (metal) chelators, unsaturated fatty acids and derivatives thereof, vitamin C and derivatives, tocopherols and derivatives, vitamin A and derivatives, rutinic acid and derivatives thereof, flavonoids and derivatives thereof, ferulic acid, furfurylideneglucitol, carnosine, butylhydroxytoluene, butylhydroxyanisole, nordohydroguaiaretic acid, trihydroxybutyrophenone, uric acid and derivatives thereof, mannose and derivatives thereof, stilbenes and derivatives thereof, derivatives of benzylidene-2,4-alkanedione, derivatives of benzylidenemalonic acid or derivatives of benzylmalonic acid is enhanced.

5. The method according to claim 1, wherein the efficacy of the compounds of the formula (A) and/or (B) is enhanced, 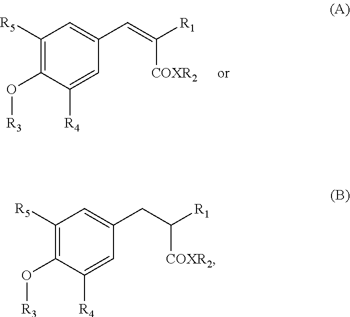
in which
R₁ denotes —C(O)R₆, —CO₂R₆ or —C(O)N(R₇)₂,
R₂ denotes linear or branched alkyl having 1 to 30 C atoms,
X denotes O, NH or a bond,
R₃ denotes H or linear or branched alkyl having 1 to 20 C atoms,
R₄ and R₅ each, independently of one another, denote H, linear or branched alkyl having 1 to 12 C atoms or linear or branched alkoxy having 1 to 12 C atoms,
R₆ denotes linear or branched alkyl having 1 to 30 C atoms and
R₇ in each case, independently of one another, denotes H or linear or branched alkyl having 1 to 8 C atoms.

6. The method according to claim 1, wherein the efficacy of the compounds bis(2-ethylhexyl) 2-(4-hydroxy-3,5-dimethoxybenzylidene)malonate, bis(2-ethylhexyl) 2-(4-hydroxy-3,5-dimethoxybenzyl)malonate, bisisopropyl 2-(4-hydroxy-3-methoxybenzylidene)malonate, bisisopropyl 2-(4-hydroxy-3-methoxybenzyl)malonate, bis(2-ethylhexyl) 2-(4-methoxybenzylidene)malonate and/or 3-(3,4,5-trimethoxybenzylidene)-2,4-pentanedione is enhanced.

7. Mixture comprising a compound of the formula (I) 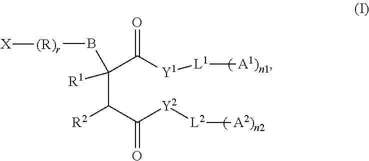
where
X is a hydrophilic group,
R is linear or branched alkylene having 1 to 12 C atoms, where one or more non-adjacent C atoms may be replaced by O, S and/or N,
r is 0 or 1,
B is a single bond, —O—, —NH—, —NR′—, —CH₂—, —C(O)—O—, —S—, —CH₂—O—, —O—C(O)—, —O—C(O)—O—, —N—C(O)—, —C(O)—N—, —O—C(O)—N—, —N—C(O)—N—, —Si(R′)₂—, —Si(R′)₂—O—, —O—SO₂— or —SO₂—O—,
R′ is a linear or branched alkyl group having 1 to 12 C atoms,
R¹ and R², independently of one another, are hydrogen or —CH₂—COY³-L³-(A³)ₙ₃,
Y¹, Y² and Y³, independently of one another, are O, S or N,
L¹, L² and L³, independently of one another, are linear or branched alkylene having 1 to 12 C atoms, where one or more non-adjacent C atoms may be replaced by O, S and/or N,
A¹, A² and A³, independently of one another, are hydrogen or a group of the structure —Zⁱ(CR³R⁴)ₘᵢRfⁱ, where i denotes 1, 2 or 3,
Zⁱ is O, S or N and is bonded to a C atom of Lⁱ or is a single bond,
R³ and R⁴, independently of one another, are hydrogen or a straight-chain or branched alkyl group having 1 to 12 C atoms,
Rfⁱ is a fluorine-containing radical,
n1, n2 and n3, independently of one another, are 1, 2 or 3,
m1, m2 and m3, independently of one another, are 0, 1, 2, 3, 4 or 5,
and the compounds of the formula (I) contain at least one Rfⁱ group, and
at least one antioxidant.

8. Mixture according to claim 7, where the at least one antioxidant corresponds to a compound of the formula (A) and/or (B), 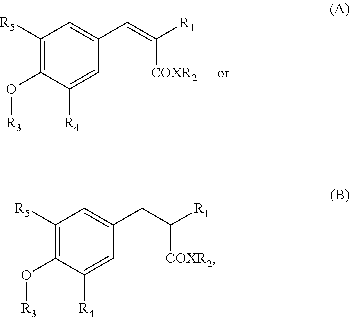
in which
R₁ denotes —C(O)R₆, —CO₂R₆ or —C(O)N(R₇)₂,
R₂ denotes linear or branched alkyl having 1 to 30 C atoms,
X denotes O, NH or a bond,
R₃ denotes H or linear or branched alkyl having 1 to 20 C atoms,
R₄ and R₅ each, independently of one another, denote H, linear or branched alkyl having 1 to 12 C atoms or linear or branched alkoxy having 1 to 12 C atoms,
R₆ denotes linear or branched alkyl having 1 to 30 C atoms and
R₇ in each case, independently of one another, denotes H or linear or branched alkyl having 1 to 8 C atoms.

9. Mixture according to claim 7, where the compound of the formula (A) and/or of the formula (B) is selected from the group bis(2-ethylhexyl) 2-(4-hydroxy-3,5-dimethoxybenzylidene)malonate, bis(2-ethylhexyl) 2-(4-hydroxy-3,5-dimethoxybenzyl)malonate, bisisopropyl 2-(4-hydroxy-3-methoxybenzylidene)malonate, bisisopropyl 2-(4-hydroxy-3-methoxybenzyl)malonate, bis(2-ethylhexyl) 2-(4-methoxybenzylidene)malonate and/or 3-(3,4,5-trimethoxybenzylidene)-2,4-pentanedione.

10. Mixture according to claim 7, comprising bis(2-ethylhexyl) 2-(4-hydroxy-3,5-dimethoxybenzylidene)malonate and/or bis(2-ethylhexyl) 2-(4-hydroxy-3,5-dimethoxybenzyl)malonate and the compound of the formula (III-1):

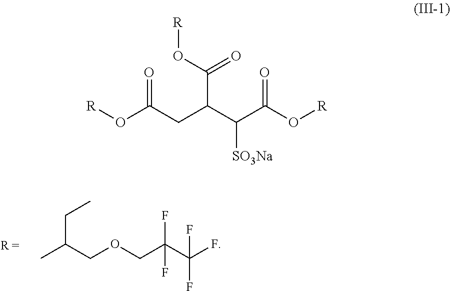

11. Cosmetic or dermatological composition or medicinal product comprising at least one compound of the formula (I) according to claim 1 and at least one antioxidant.

12. The composition or product according to claim 11, where the at least one antioxidant corresponds to the compound of the formula (A) and/or (B) 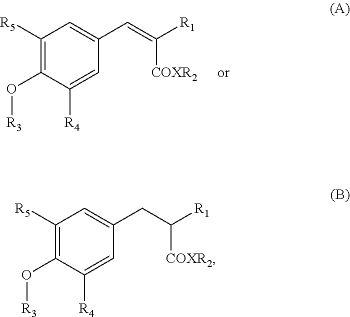
in which
R₁ denotes —C(O)R₆, —CO₂R₆ or —C(O)N(R₇)₂,
R₂ denotes linear or branched alkyl having 1 to 30 C atoms,
X denotes O, NH or a bond,
R₃ denotes H or linear or branched alkyl having 1 to 20 C atoms,
R₄ and R₅ each, independently of one another, denote H, linear or branched alkyl having 1 to 12 C atoms or linear or branched alkoxy having 1 to 12 C atoms,
R₆ denotes linear or branched alkyl having 1 to 30 C atoms and
R₇ in each case, independently of one another, denotes H or linear or branched alkyl having 1 to 8 C atoms.

13. The composition or product according to claim 11, comprising at least one organic or inorganic UV filter.

14. The composition or product according to claim 11, comprising a cosmetic or dermatological vehicle or a vehicle which is tolerated for medicinal products.

15. Process for the preparation of a composition or product according to claim 11, comprising mixing together at least one compound of the formula (I) and at least one antioxidant and optionally a vehicle, an organic and/or inorganic UV filter and further active substances or assistants.
